(19) United States
(12) Patent Application Publication
(10) Pub. No.: US 2026/0255868 A1
(43) Pub. Date: Aug. 27, 2026

(54) TETRADENTATE PLATINUM COMPLEX LIGHT-EMITTING MATERIAL CONTAINING SPIRO STRUCTURE AND USE THEREOF

(71) Applicant: GUANGDONG AGLAIA OPTOELECTRONIC MATERIALS CO., LTD, Foshan, Guangdong (CN)

(72) Inventors: Huiyang LI, Foshan, Guangdong (CN); Xinwei WU; Haijian TAN; Lei DAI, Foshan, Guangdong (CN); Lei CAI, Foshan, Guangdong (CN)

(73) Assignee: GUANGDONG AGLAIA OPTOELECTRONIC MATERIALS CO., LTD, Foshan, Guangdong (CN)

(21) Appl. No.: 18/857,799

(22) PCT Filed: Apr. 14, 2023
(86) PCT No.: PCT/CN2023/088243

§ 371 Date: Oct. 17, 2024

(30) Foreign Application Priority Data
Apr. 10, 2022 (CN) 202310375613.2
Apr. 19, 2022 (CN) 202210412062.8

(51) Int. Cl. H10K 85/30; C07D 401/14; C07D 403/10; C07F 15/00; C09K 11/06; H10K 50/12

(52) U.S. Cl. CPC H10K 85/346; C07D 401/14; C07D 403/10; C07F 15/0086; C09K 11/06; C09K 2211/1007; C09K 2211/1011; C09K 2211/1029; C09K 2211/1044; C09K 2211/185; H10K 50/12

(57) ABSTRACT

A tetradentate platinum complex containing a spiro structure and use thereof in an organic light-emitting diode. The tetradentate platinum complex is a compound with a structure of chemical formula (I), and such material has a relatively high luminescence quantum yield and a short excited state lifetime. When applied to an organic light-emitting diode, the material has good luminous efficiency and device stability. The present disclosure also relates to an organic electroluminescent device, which includes a cathode, an anode and an organic layer. The organic layer is one or more of a hole injection layer, a hole transport layer, a luminescent layer, a hole blocking layer, an electron transport layer and an electron injection layer, and at least one of the organic layer contains the compound of structural formula (I).

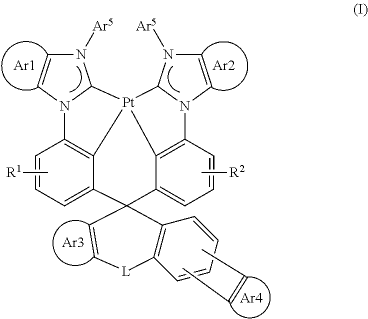

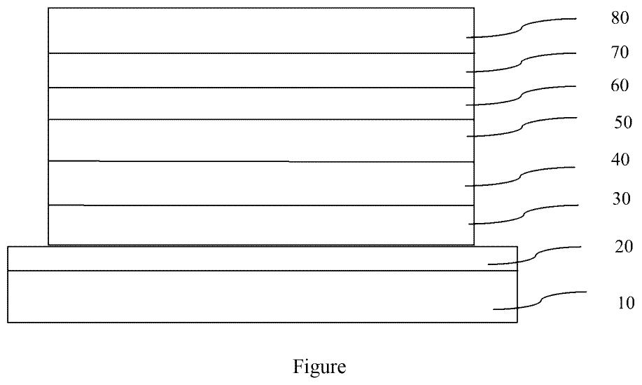

### CROSS-REFERENCE TO RELATED APPLICATIONS

The present application is a national phase entry under 35 USC § 371 of International Application No. PCT/CN2023/088243 filed Apr. 14, 2023, which claims the benefit of and priority to Chinese Patent Application No. 202210412062.8, filed Apr. 19, 2022, and Chinese Patent Application No. 202310375613.2, filed Apr. 10, 2023, the entire disclosures of which are incorporated herein by reference.

### TECHNICAL FIELD

The present disclosure relates to the field of electroluminescent materials, and in particular, to a tetradentate platinum complex containing a spiro structure and use thereof in an organic light-emitting diode.

### BACKGROUND

Organic light-emitting diode (OLED) has attracted widespread attention from academia and industry and has become one of the focuses of competition in the high-tech fields of various countries, due to many advantages such as self-illumination, wide color gamut, wide viewing angle, and easy implementation of flexible displays. However, the existing OLED still has low efficiency, short service life and other disadvantages, thus further research is required.

The luminous efficiency and stability of OLED devices depend largely on the light-emitting materials used. Early fluorescent OLED may usually only use singlet-state excitons to emit light, triplet-state excitons generated in the device cannot emit light and return to the ground state through non-radiation, which hinders the improvement of OLED efficiency. In 1998, Professor Zhiming ZHI of the University of Hong Kong and his collaborators used transition metal complexes to achieve triplet-state luminescence, effectively improving the exciton utilization rate. In the same year, Thompson et al. also reported the electrophosphorescence phenomenon of transition metal complexes. Phosphorescent OLED may effectively utilize triplet-state and singlet-state excitons and can theoretically achieve 100% internal quantum efficiency, which promotes the commercialization process of OLED. The control of OLED emitting color may be achieved through the structural design of the light-emitting materials. OLED may include one light-emitting layer or multiple light-emitting layers to achieve the desired spectrum. Green, yellow and red phosphorescent materials have been commercialized. Commercial OLED displays usually use blue fluorescence and yellow, or green and red phosphorescence to achieve full-color display. The light-emitting materials with higher efficiency and longer service life are urgently needed by the current industry.

Metal complex light-emitting materials have been used in OLED products, but their performance, such as luminous efficiency and service life of device, still need to be further improved. At present, blue transition metal complexes have made breakthroughs in luminous efficiency, but the service life of device is short. Therefore, the development of efficient and stable blue phosphorescent materials has the practical application value.

### SUMMARY

For the above problems, the present disclosure provides a tetradentate platinum complex light-emitting material containing a spiro structure. Such material is applied to organic light-emitting diodes to exhibit good light-emitting performance and service life of device.

The present disclosure also provides an organic light-emitting diode based on the platinum complex.

The tetradentate platinum complex containing a spiro structure is a compound with a structure of formula (I):

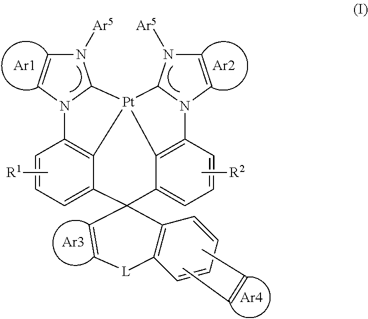

wherein:
L is selected from CR³R⁴, NR⁵, O, S or a single bond;
R¹ to R⁵ are independently selected from hydrogen, deuterium, halogen, amine, carbonyl group, carboxyl group, cyano group, phosphino, substituted or unsubstituted alkyl group with 1-20 carbon atoms, substituted or unsubstituted cycloalkyl group with 3-20 ring carbon atoms, substituted or unsubstituted alkenyl group with 2-20 carbon atoms, substituted or unsubstituted alkoxy group with 1-20 carbon atoms, substituted or unsubstituted aryl group with 6-30 carbon atoms, or substituted or unsubstituted heteroaryl group with 3-30 carbon atoms;
Ar¹ to Ar⁵ are independently selected from substituted or unsubstituted aromatic ring with 6-30 carbon atoms, or substituted or unsubstituted heteroaromatic ring with 3-30 carbon atoms;
a substitution is conducted by halogen, amine, cyano group or C1-C4 alkyl group; and
a heteroatom in the heteroaryl group or the heteroaromatic ring is at least one of N, S, and O.

In an embodiment, R¹ to R⁵ are each independently selected from hydrogen, deuterium, halogen, amine, cyano group, substituted or unsubstituted alkyl group with 1-6 carbon atoms, substituted or unsubstituted cycloalkyl group with 3-6 ring carbon atoms, substituted or unsubstituted alkenyl group with 2-6 carbon atoms, substituted or unsubstituted alkoxy group with 1-6 carbon atoms, substituted or unsubstituted aryl group with 6-12 carbon atoms, or substituted or unsubstituted heteroaryl group with 3-6 carbon atoms; and
Ar¹ to Ar⁵ are independently selected from substituted or unsubstituted aromatic ring with 6-12 carbon atoms, or substituted or unsubstituted heteroaromatic ring with 3-12 carbon atoms.

In an embodiment, R¹ to R⁵ are each independently selected from hydrogen, deuterium, halogen, cyano group, C1-C4 alkyl group, substituted or unsubstituted cycloalkyl group with 3-6 ring carbon atoms, substituted or unsubstituted aryl group with 6-12 carbon atoms, or substituted or unsubstituted heteroaryl group with 3-6 carbon atoms; and
Ar¹ to Ar⁵ are independently selected from substituted or unsubstituted aromatic ring with 6-12 carbon atoms, or substituted or unsubstituted heteroaromatic ring with 3-12 carbon atoms.

In an embodiment, R¹ to R⁵ are each independently selected from hydrogen, deuterium, halogen, cyano group, methyl, isopropyl, isobutyl, tert-butyl, substituted or unsubstituted cyclopentyl, substituted or unsubstituted cyclohexyl, substituted or unsubstituted phenyl, substituted or unsubstituted pyridyl, substituted or unsubstituted pyrazinyl, or substituted or unsubstituted pyrimidinyl;
Ar¹ to Ar⁵ are independently selected from substituted or unsubstituted benzene ring, substituted or unsubstituted pyridine ring, substituted or unsubstituted pyrazine ring, substituted or unsubstituted pyrimidine ring, substituted or unsubstituted furan ring, substituted or unsubstituted thiophene ring, substituted or unsubstituted naphthalene ring, substituted or unsubstituted benzofuran ring, substituted or unsubstituted benzothiophene ring, substituted or unsubstituted thiazole ring, substituted or unsubstituted oxazole ring, substituted or unsubstituted pyrrole ring or substituted or unsubstituted imidazole ring; and
a substitution is conducted by cyano group or C1-C4 alkyl group.

In an embodiment, in formula (I), R¹ to R⁵ are each independently selected from hydrogen, deuterium, fluorine, chlorine, methyl, tert-butyl, cyano group, substituted or unsubstituted cyclopentyl, substituted or unsubstituted cyclohexyl, or substituted or unsubstituted phenyl; and
Ar¹ to Ar⁵ are independently selected from the substituted or unsubstituted benzene ring, the substituted or unsubstituted pyridine ring, furan ring, thiophene ring, benzothiophene ring, benzofuran ring, or pyridine ring.

In an embodiment, in formula (I), R¹ to R² are each independently selected from hydrogen, deuterium, chlorine, tert-butyl; and R³ to R⁵ are each independently selected from hydrogen, deuterium, tert-butyl, or phenyl; and
Ar¹, Ar², and Ar⁵ are independently selected from the benzene ring, benzothiophene ring and benzofuran ring, and Ar³ and Ar⁴ are selected from the benzene ring, pyridine ring, furan ring and thiophene ring.
R¹ to R² are each independently selected from hydrogen, deuterium, chlorine and tert-butyl, and
L is a single bond; and
Ar¹ and Ar² are selected from the benzene ring; Ar⁵ is selected from the benzene ring, benzothiophene ring, benzofuran ring and pyridine ring; Ar³ is selected from the benzene ring; and Ar⁴ is selected from the benzene ring, pyridine ring, furan ring and thiophene ring.

Examples of platinum metal complexes according to the present disclosure are listed below, but not limited to the listed structures:

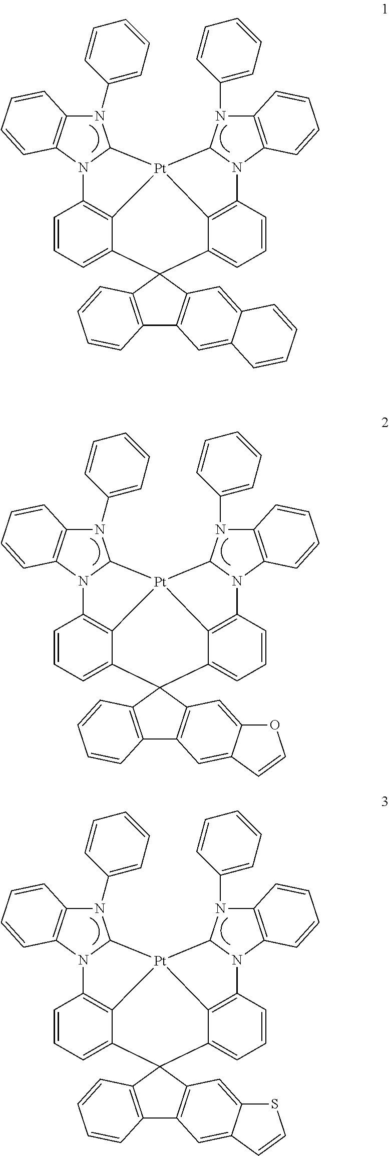

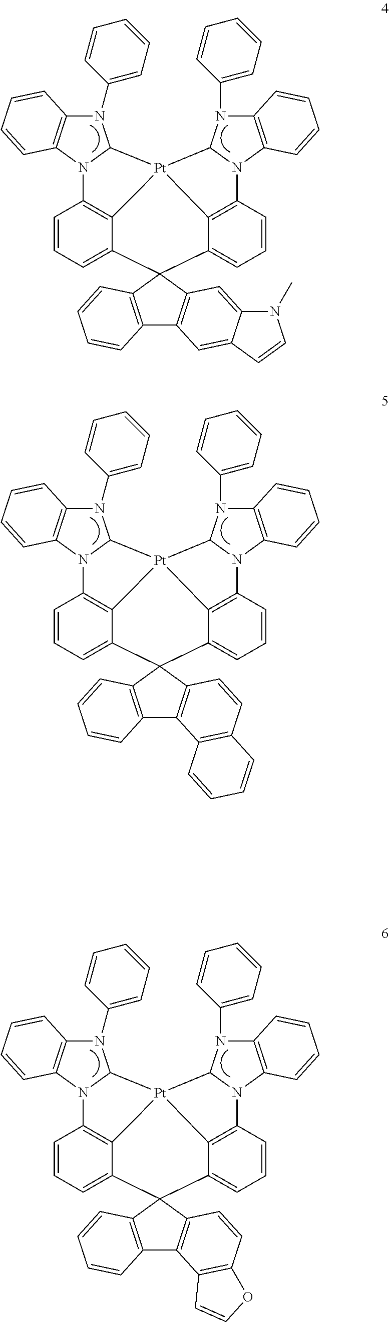

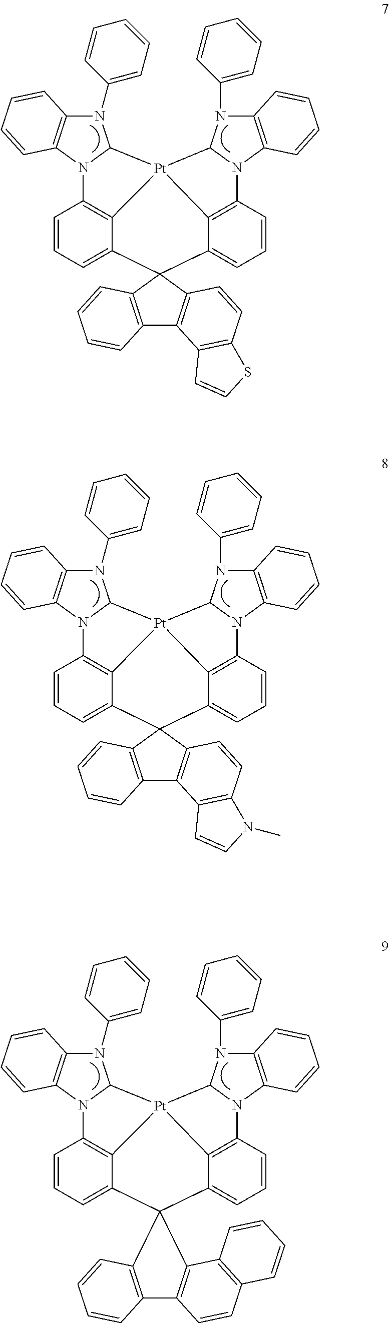

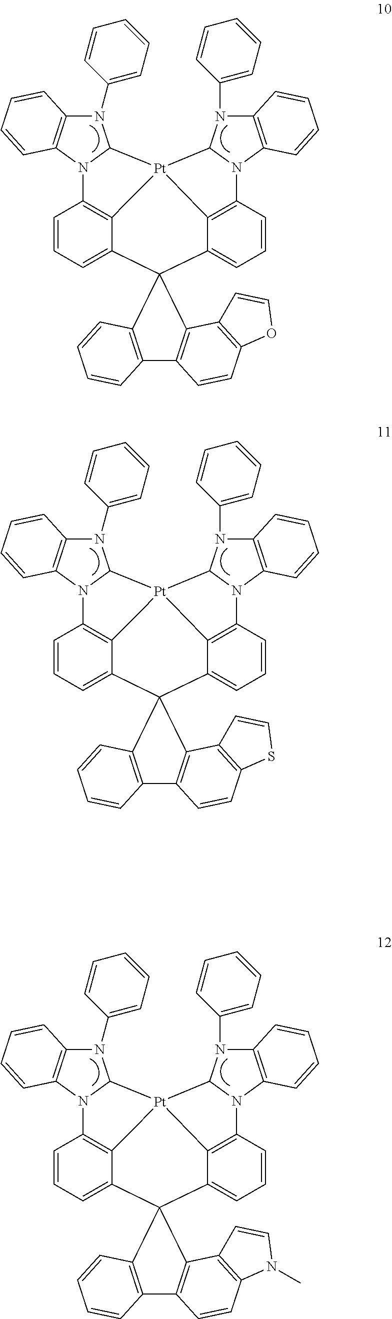

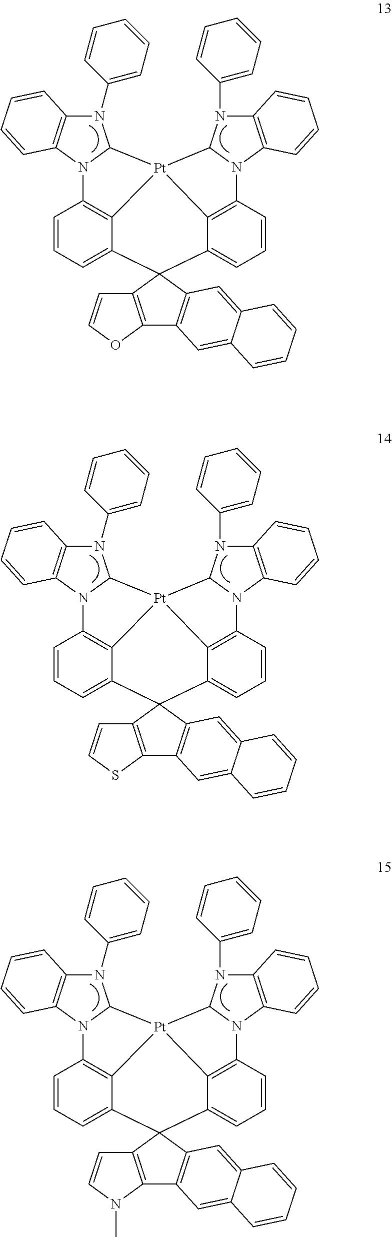

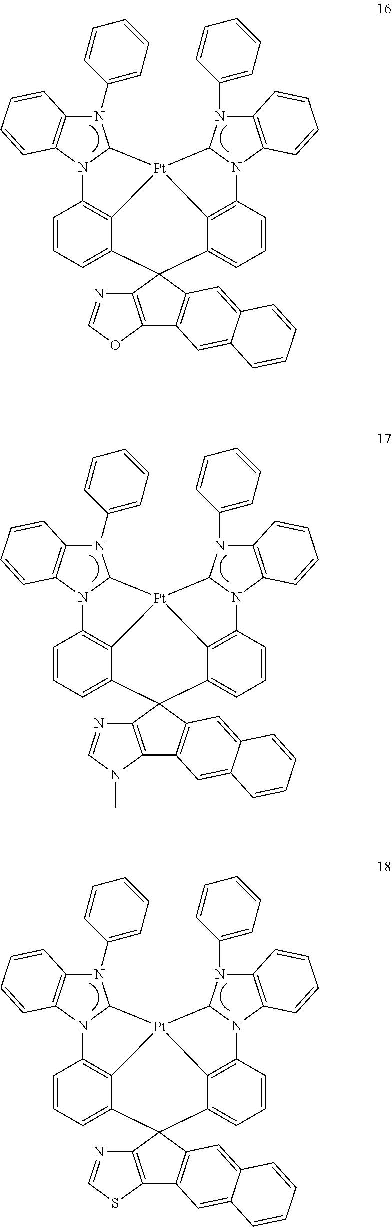

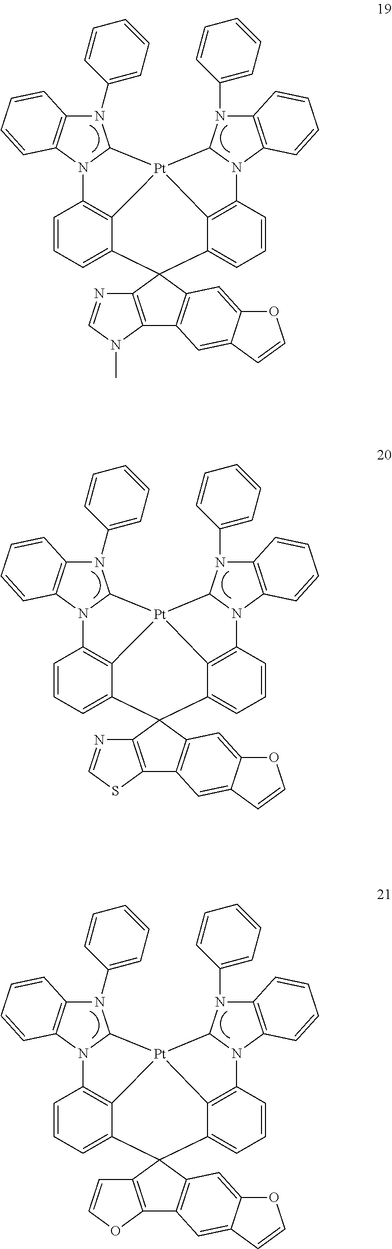

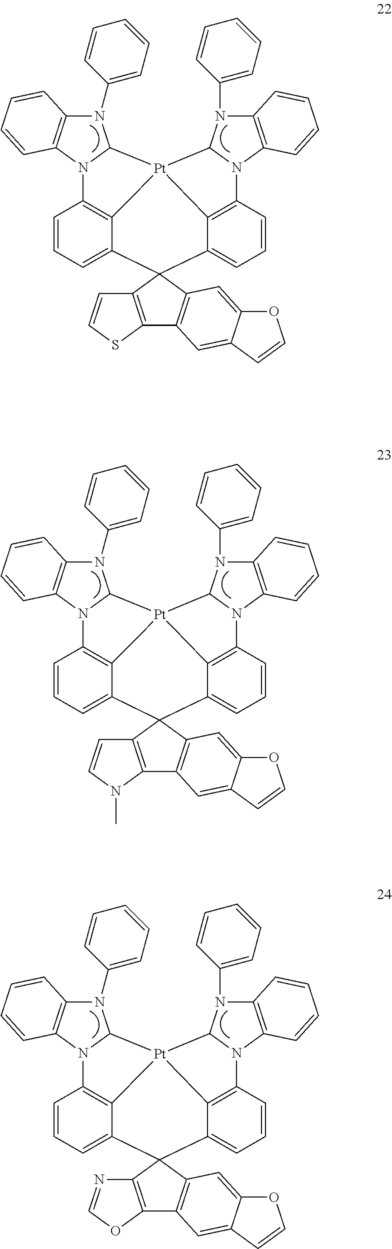

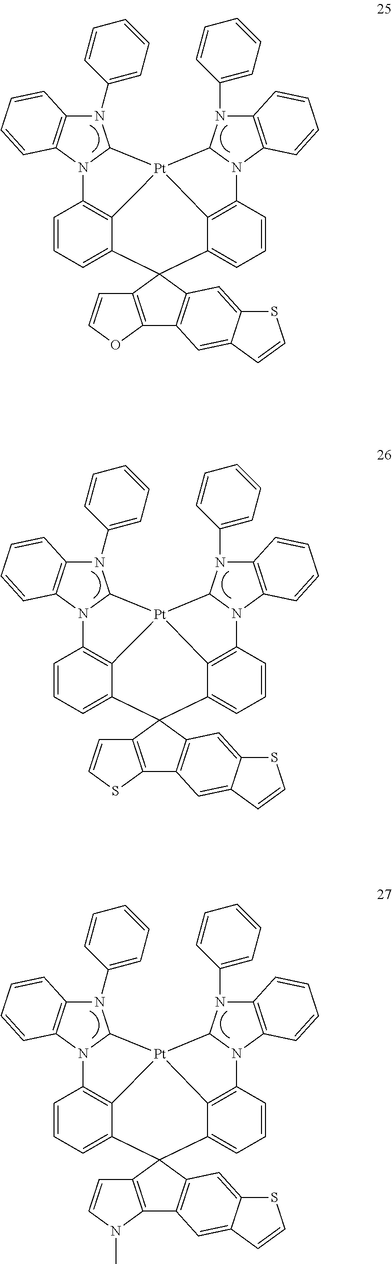

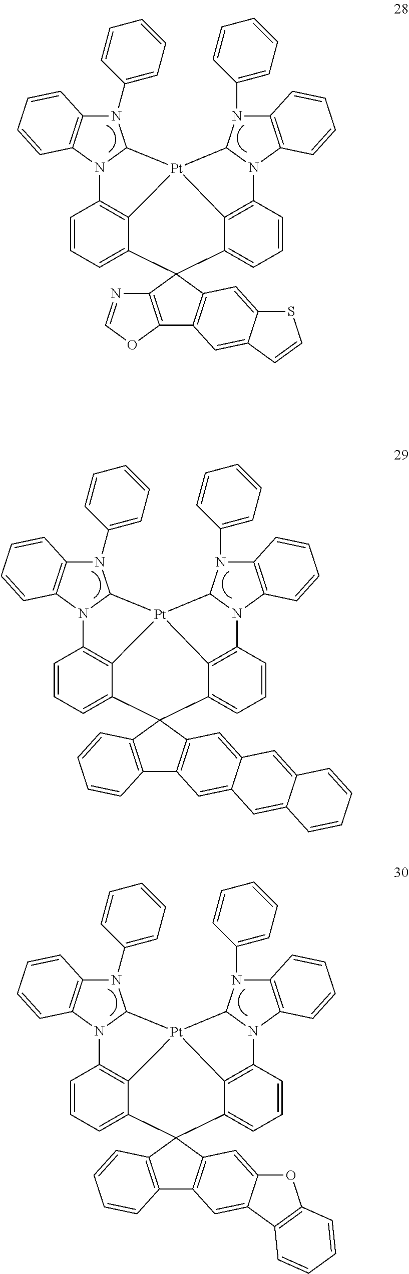

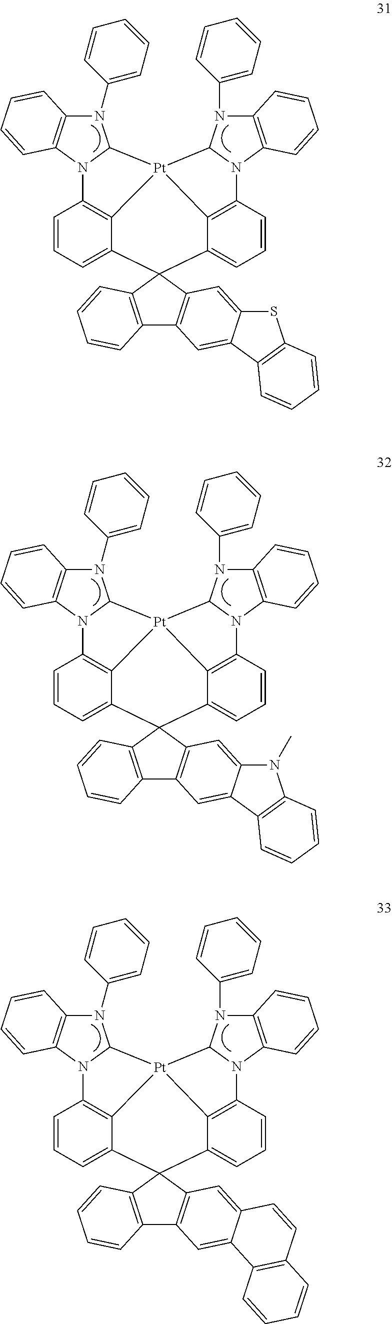

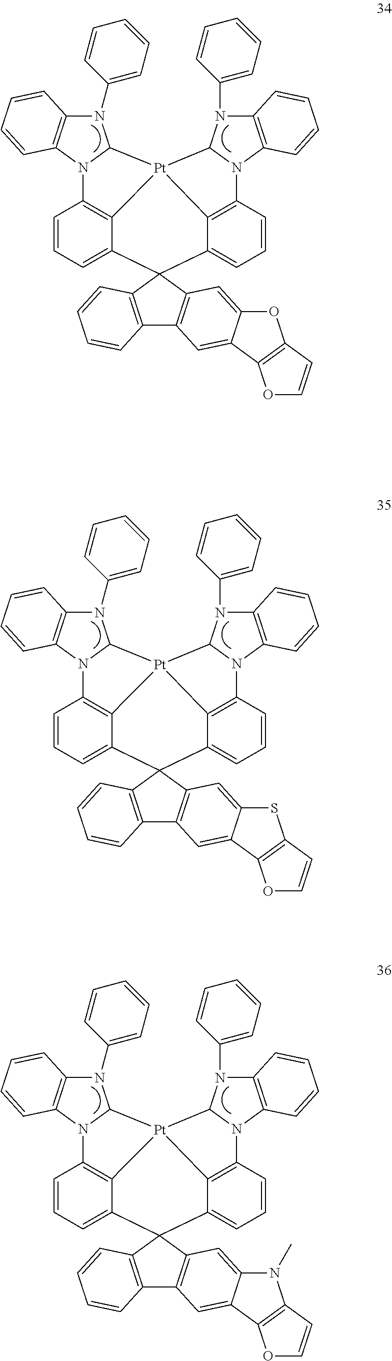

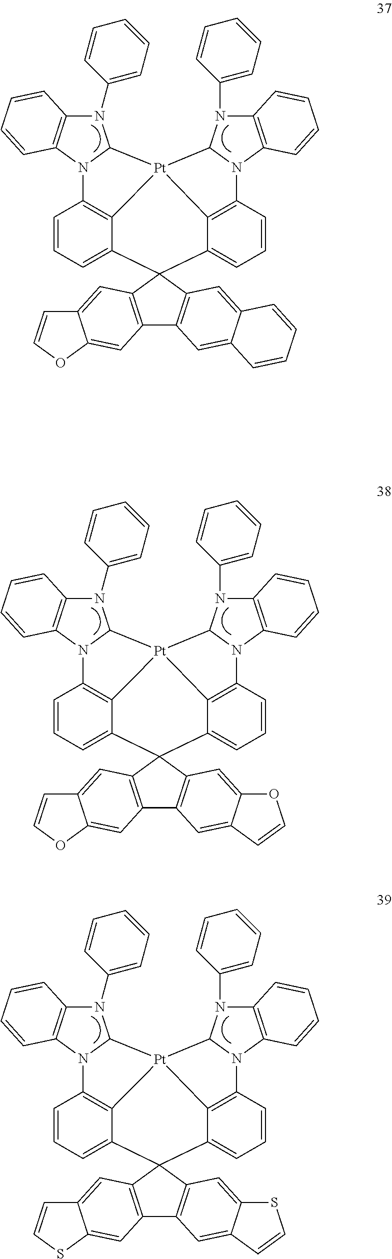

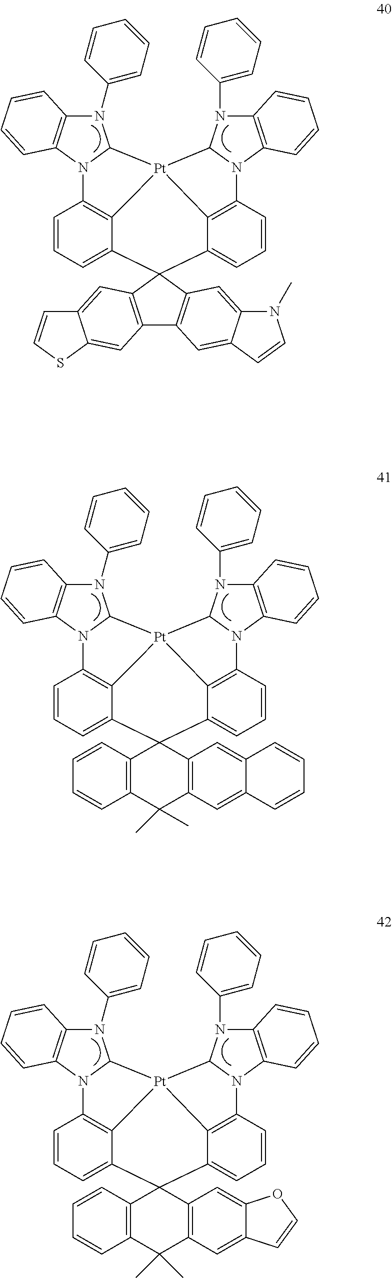

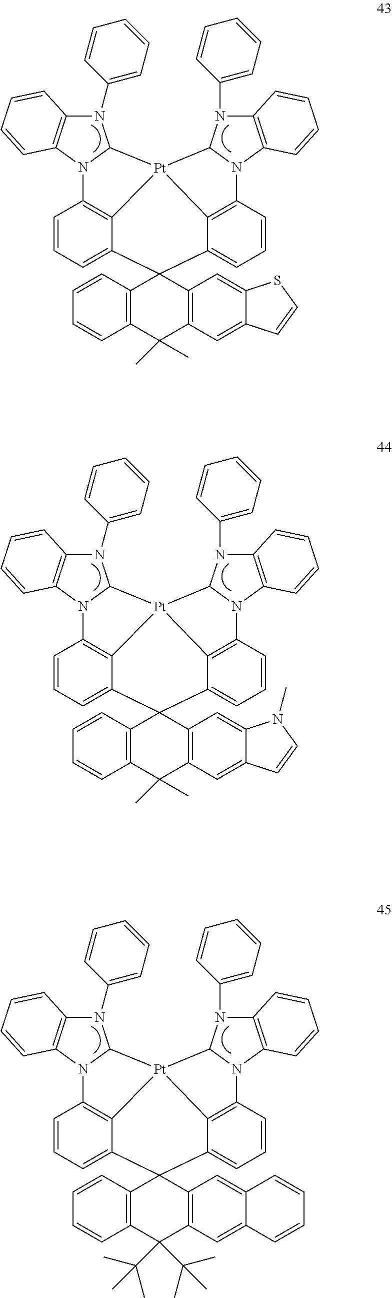

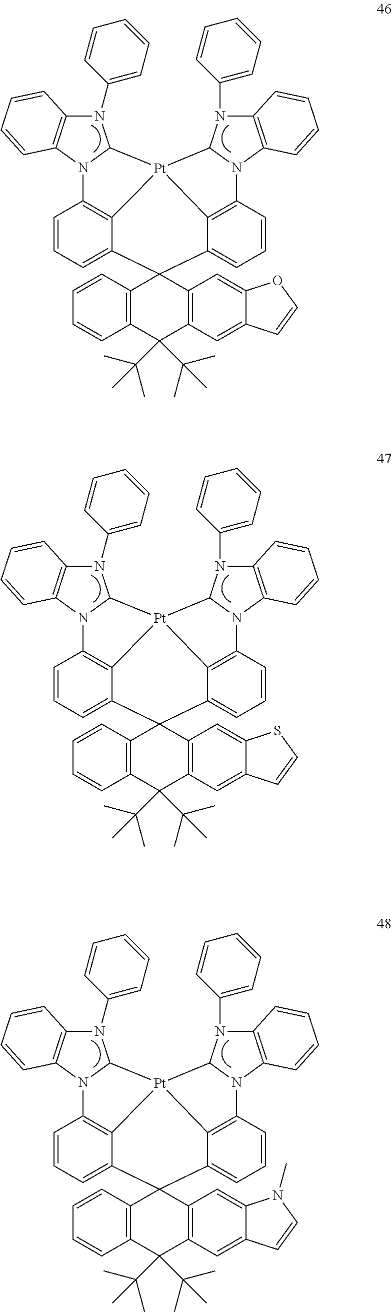

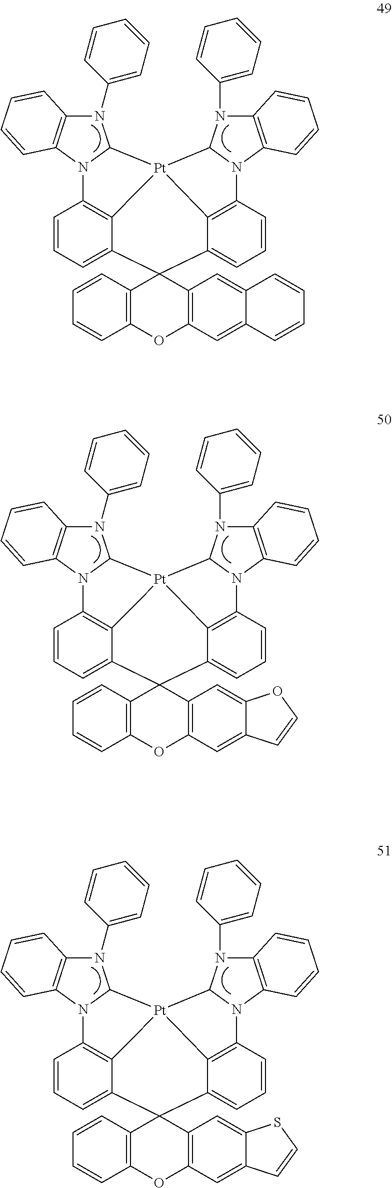

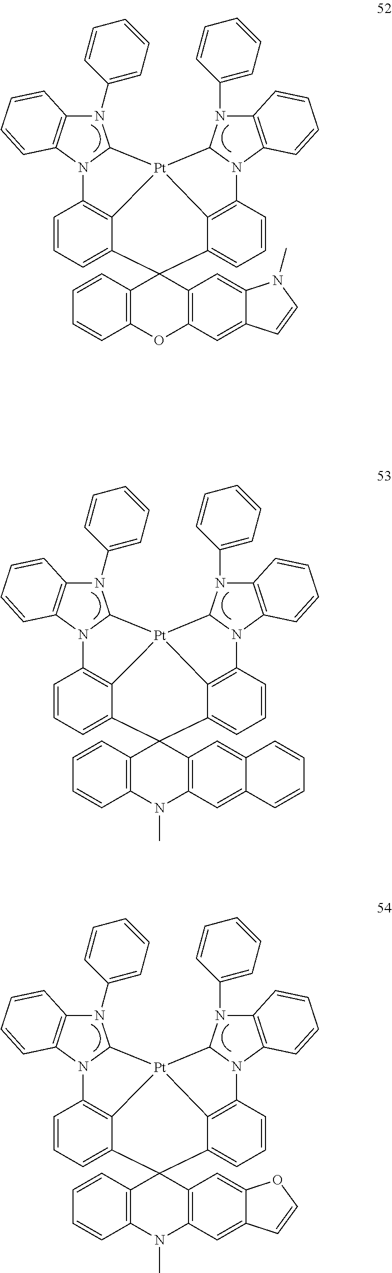

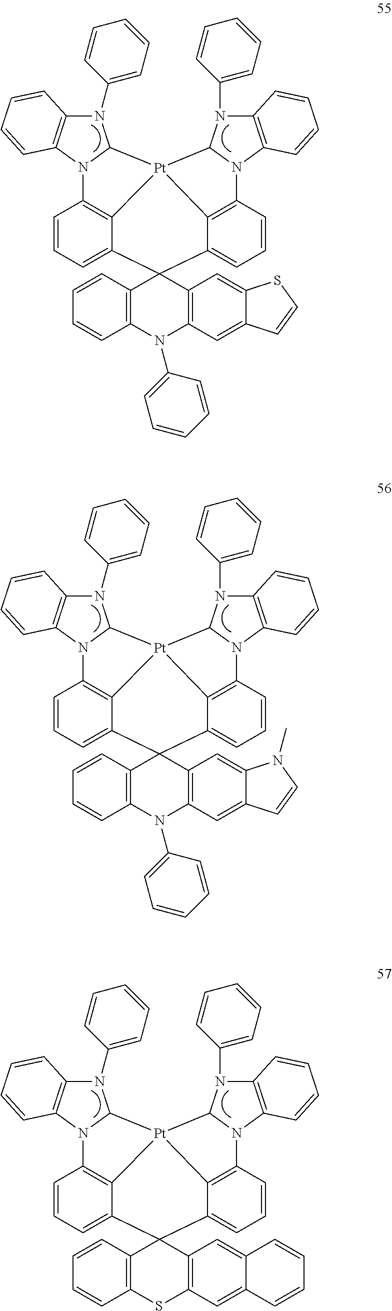

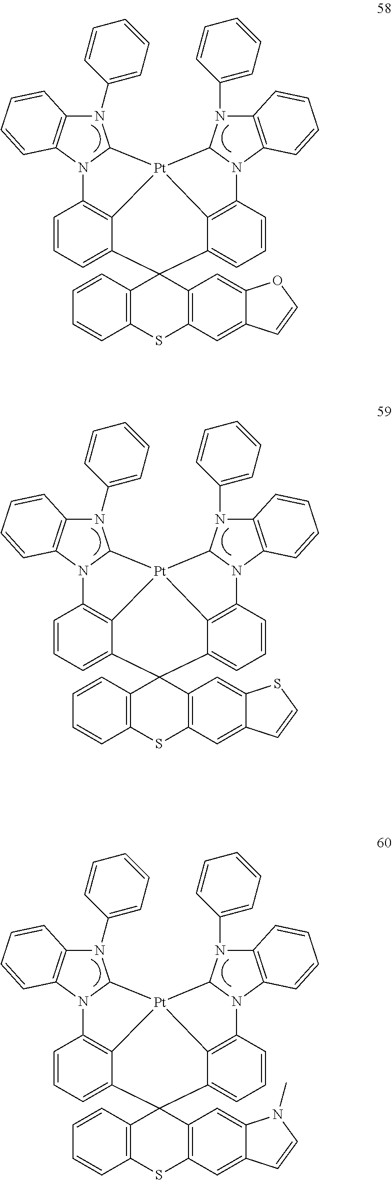

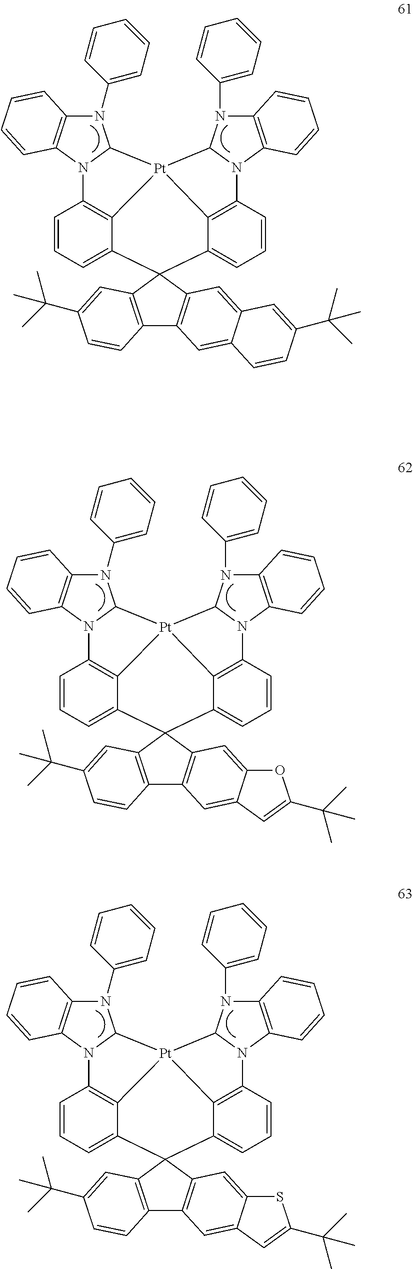

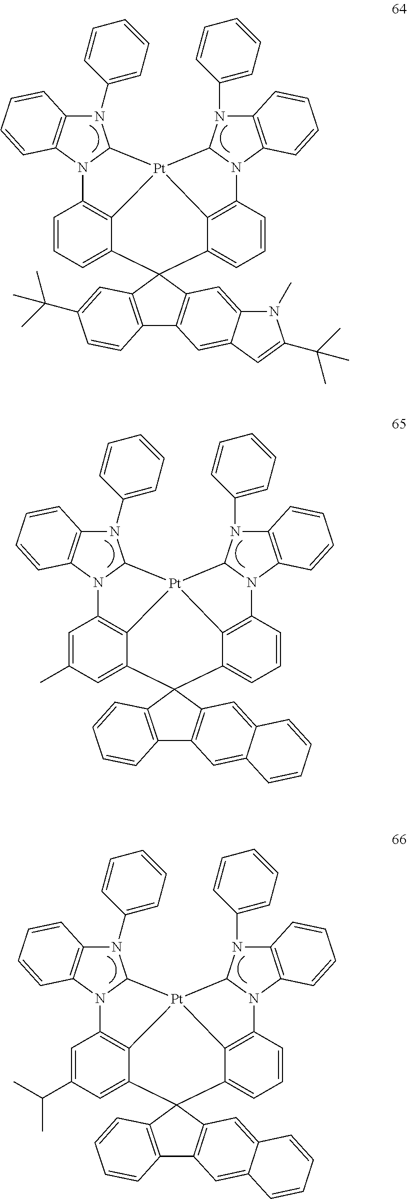

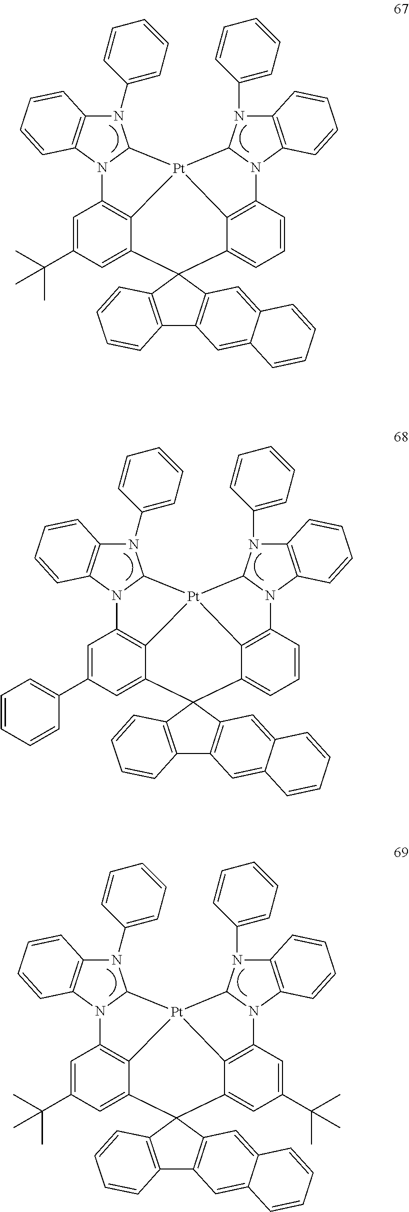

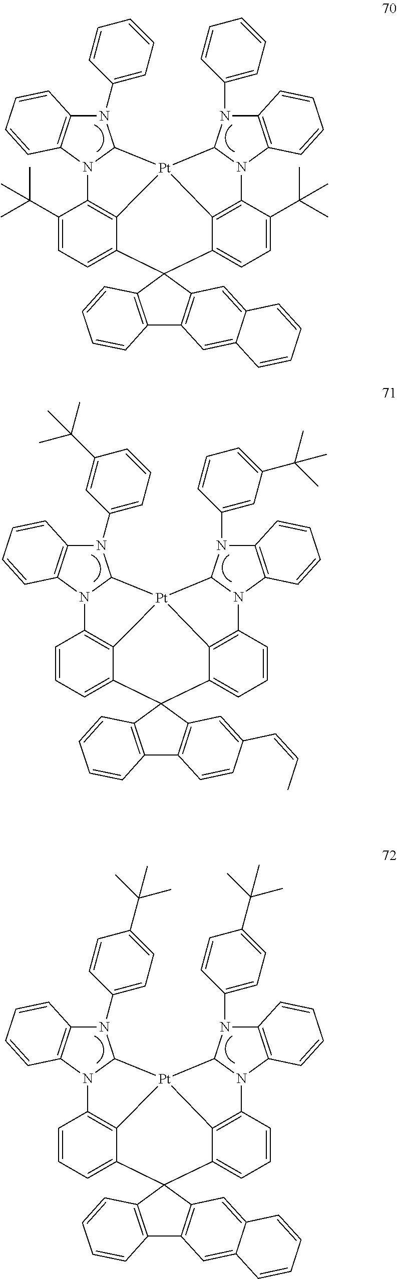

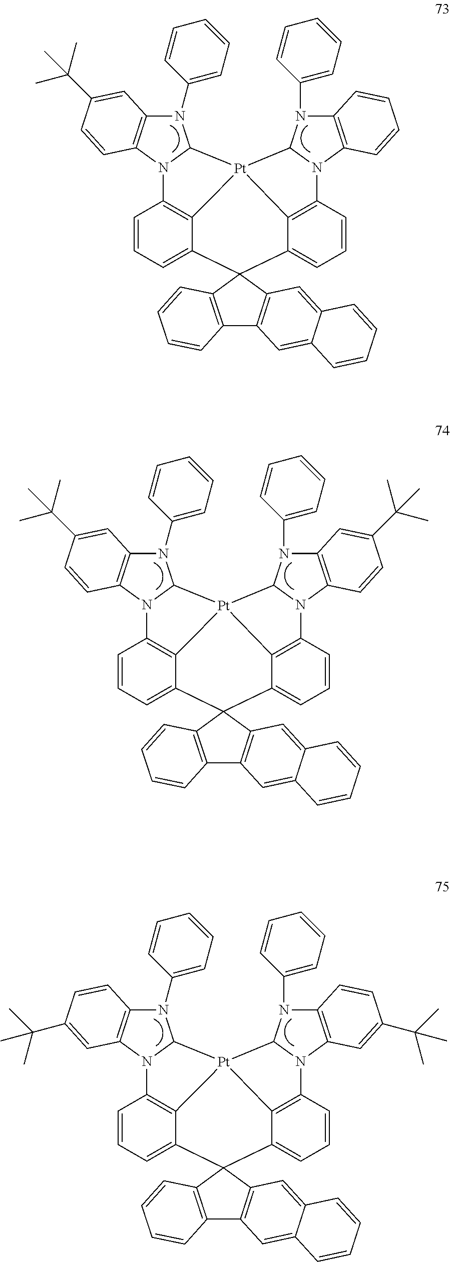

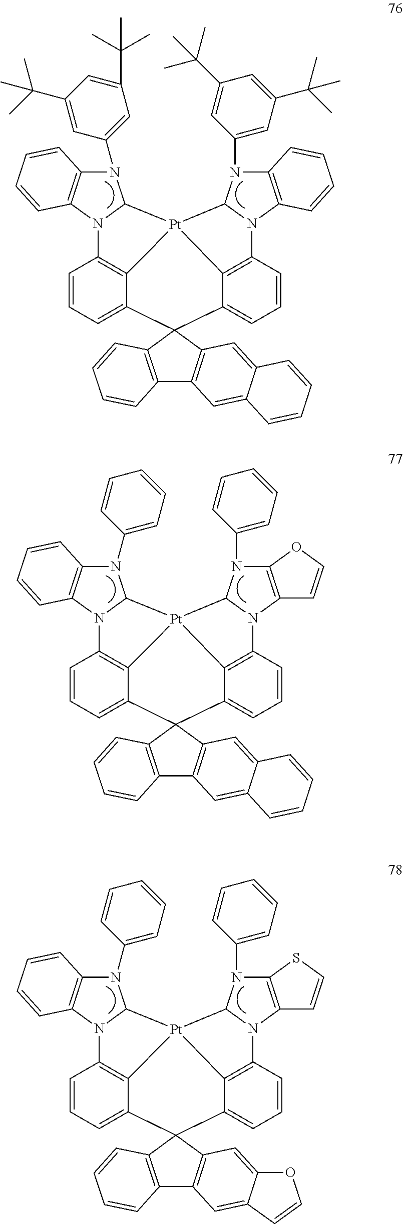

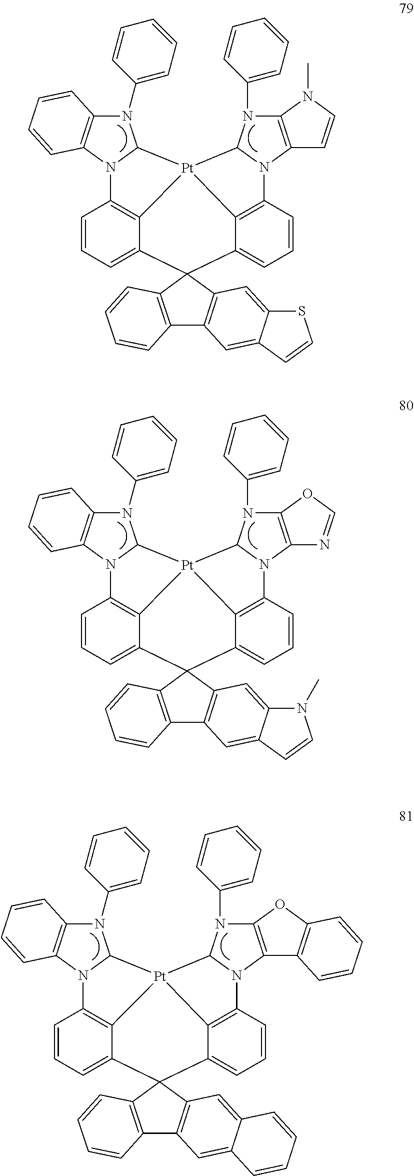

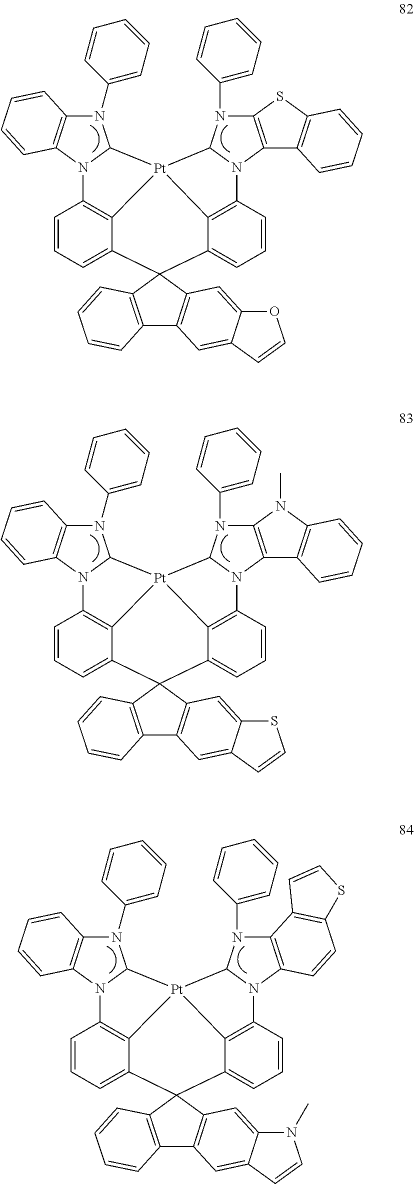

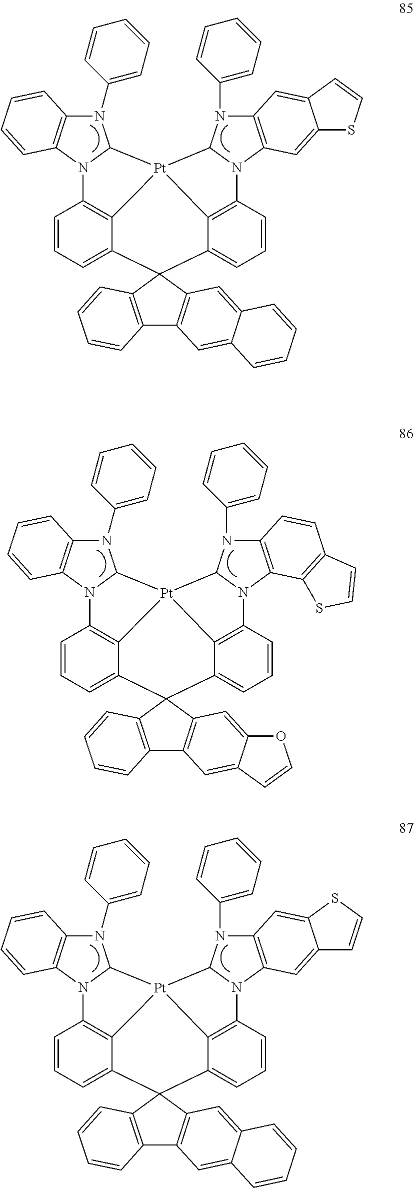

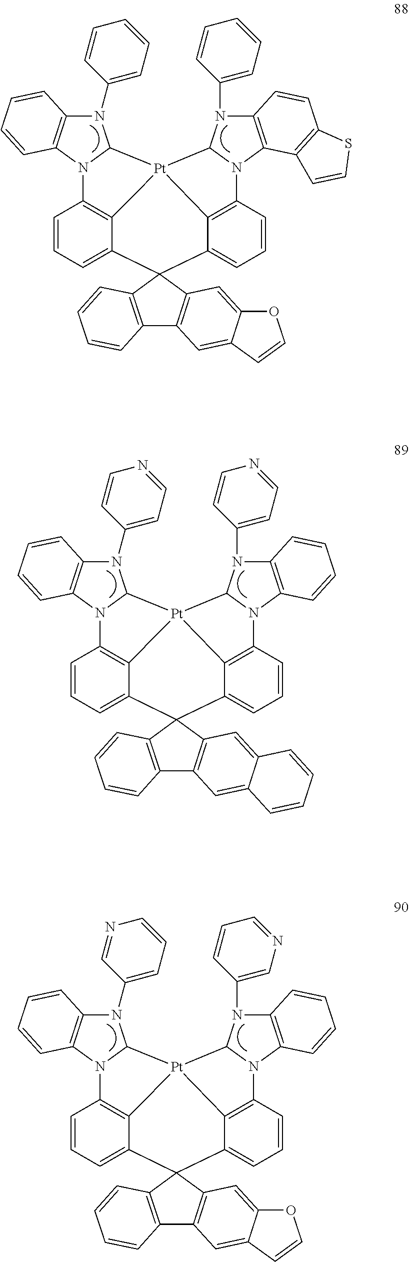

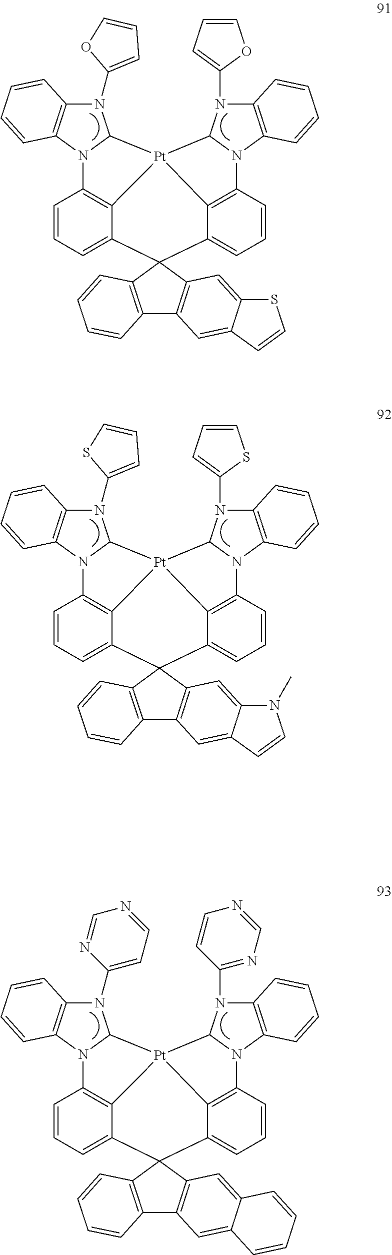

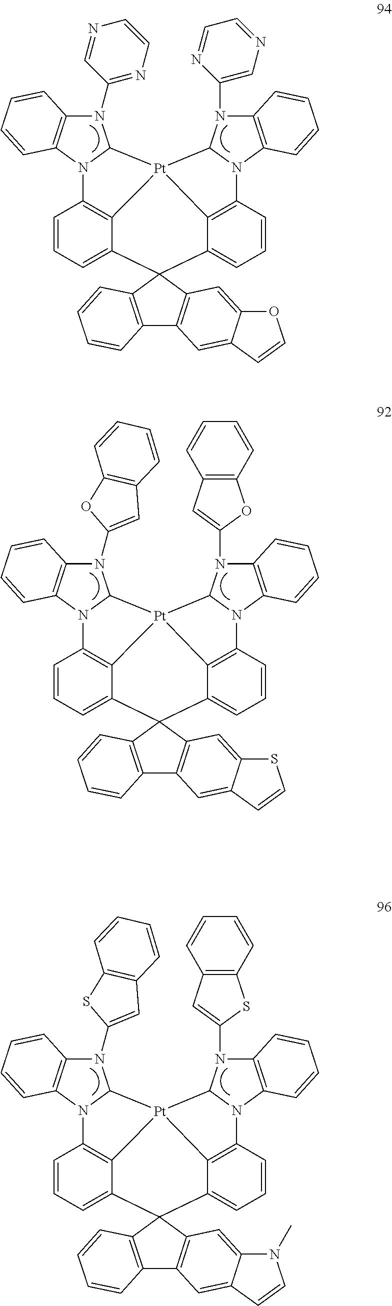

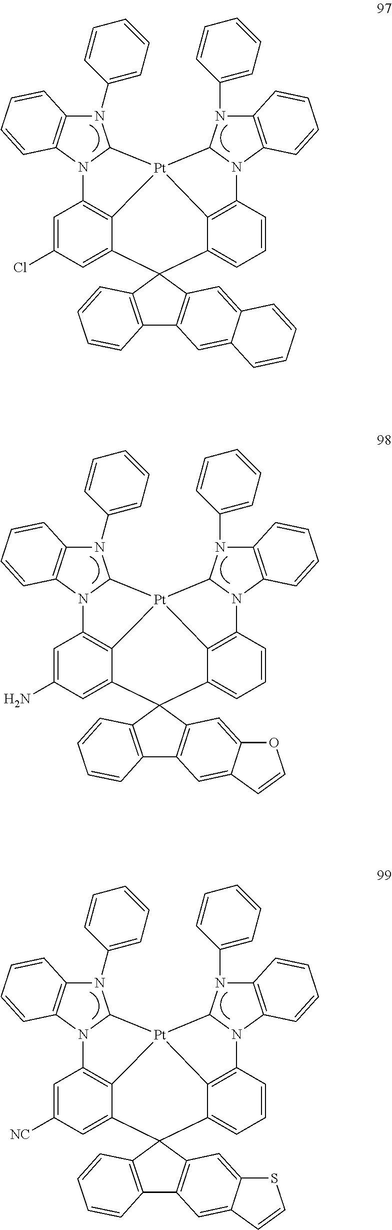

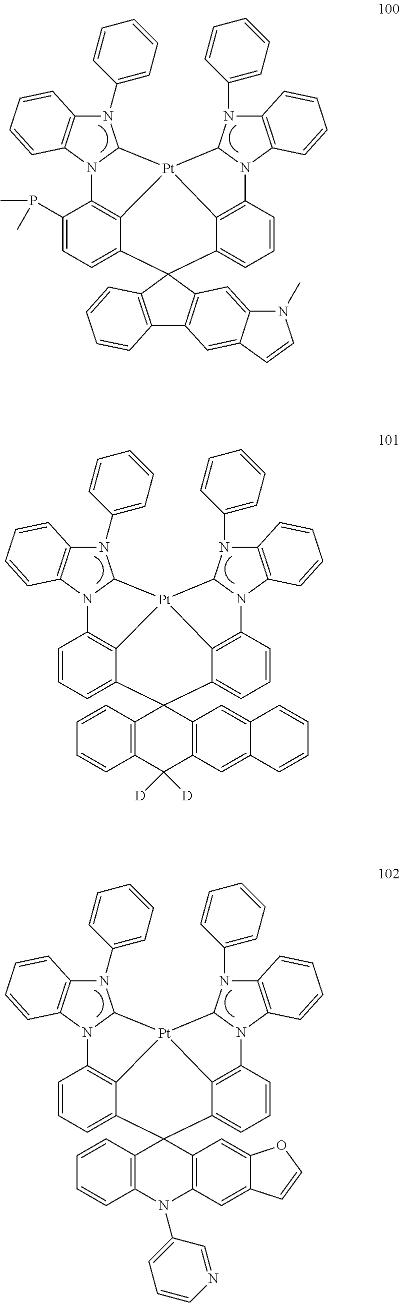

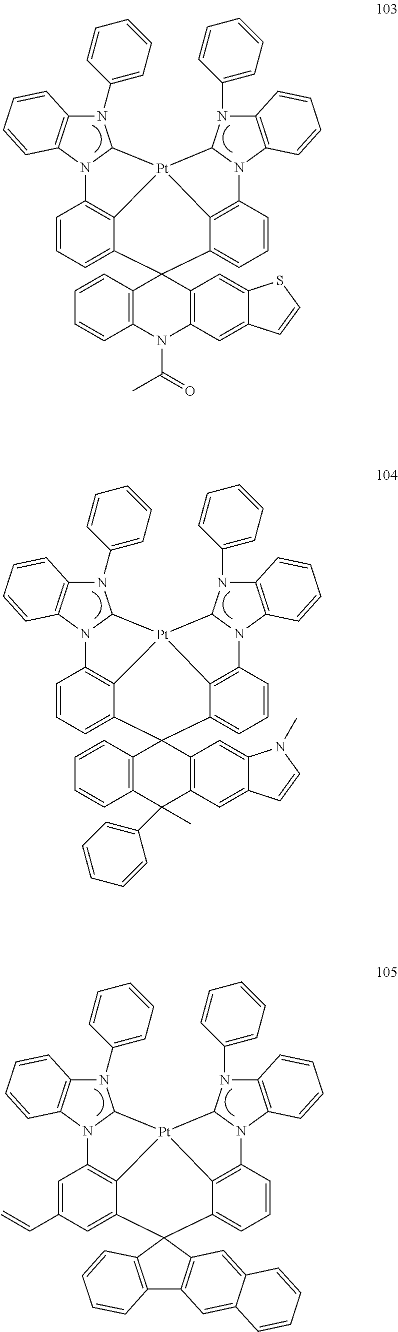

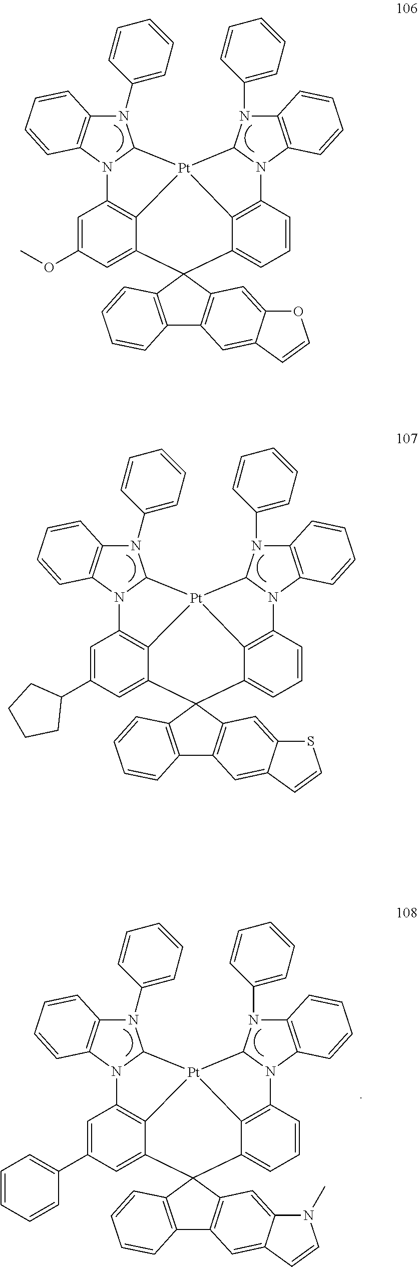

A precursor of the above metal complex has a structural formula as follows:

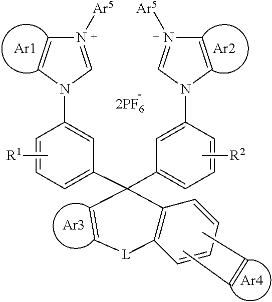

wherein L, R¹, R², Ar¹, Ar², Ar³, Ar⁴ and Ar⁵ are defined as formula (I).

The present disclosure also provides use of the above-mentioned platinum complex in organic optoelectronic devices. The optoelectronic devices include, but not limited to, organic light-emitting diode (OLED), organic thin film transistor (OTFT), organic photovoltaic device (OPV), and light-emitting electrochemical cell (LCE) and chemical sensor, such as OLED.

An organic light-emitting diode (OLED) containing the above-mentioned platinum complex, wherein the platinum complex is a light-emitting material in a light-emitting device.

The organic light-emitting diode in the present disclosure includes a cathode, an anode and an organic layer. The organic layer is one or more of a hole injection layer, a hole transport layer, a light-emitting layer, a hole blocking layer, an electron injection layer and an electron transport layer. These organic layers need not be present in every layer; at least one layer among the hole injection layer, hole transport layer, hole blocking layer, electron injection layer, light-emitting layer and electron transport layer contains the platinum complex described in formula (I).

In an embodiment, the layer in which the platinum complex described in formula (I) is located is the light-emitting layer or the electron transport layer.

The total thickness of the organic layer of the device of the present disclosure is 1-1000 nm, such as 1-500 nm, such as 5-300 nm.

The organic layer may be formed into a thin film by distillation or a solution method.

A series of tetradentate platinum complex light-emitting materials containing a spiro structure disclosed by the present disclosure show unexpected characteristics, which can effectively suppress intermolecular interactions, effectively improve the purity of light-emitting color, and have a short excited state service life, thus significantly improving the luminous efficiency and device stability and meeting the requirements of OLED panels for the light-emitting materials.

### BRIEF DESCRIPTION OF DRAWINGS

FIGURE is a structural diagram of an organic light-emitting diode device of the present disclosure.

In which, 10 represents a glass substrate, 20 represents an anode, 30 represents a hole injection layer, 40 represents a hole transport layer, 50 represents a light-emitting layer, 60 represents an electron transport layer, 70 represents an electron injection layer, and 80 represents a cathode.

### DETAILED DESCRIPTION

### Example 1: Preparation of Complex 1

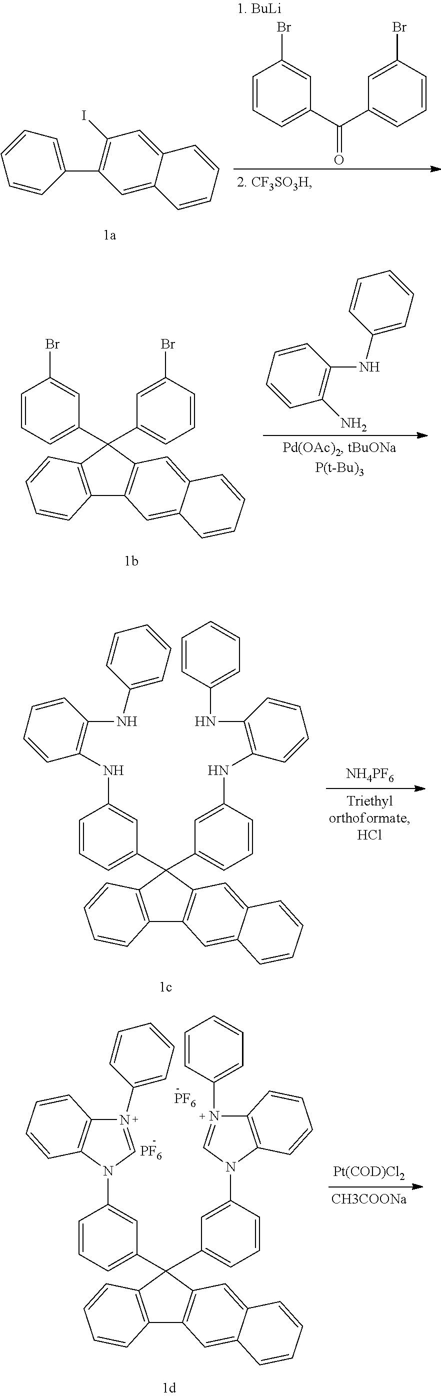

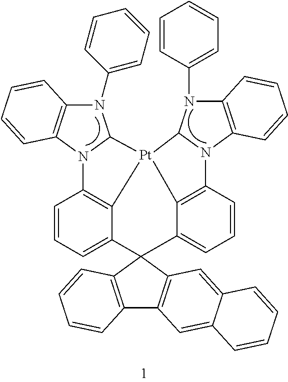

### Synthesis of Compound 1b

Under nitrogen protection, compound 1a (6.5 g, 19.7 mmol, synthesized with reference to Org. Lett. 2014, 16, 4416) was dissolved in tetrahydrofuran (50 mL), cooled to −78° C., and stirred for 0.5 h. A tetrahydrofuran solution (20 mL) with 3,3′-dibromobenzophenone (6.7 g, 19.7 mmol) was dropwise added to the above solution, stirred to react for 30 min, raised to a room temperature, and continued the reaction for 2 h. After the reaction was completed, water (50 ml) was added to quench the reaction, and extraction was carried out by ethyl acetate, and a solvent was evaporated under a reduced pressure to obtain a light yellow solid. The solid was dissolved in dichloromethane (100 mL) at 0° C., added with trifluoromethanesulfonic acid (2.0 g), and naturally heated to a room temperature and stirred overnight. After the end of the reaction, the pH was adjusted to 7-8, extraction was carried out with dichloromethane, an organic phase was evaporated under a reduced pressure, a crude product was recrystallized with dichloromethane/methanol to obtain 5.9 g of white solid, with a yield of 57%. HRMS (ESI) (m/z): 526.9842 [M+H]⁺.

### Synthesis of Compound 1c

Under nitrogen protection, a mixture of 1b (4.6 g, 8.7 mmol), sodium tert-butoxide (2.5 g, 26.2 mmol), N-phenyl o-phenylenediamine (4.0 g, 21.9 mmol), palladium acetate (0.2 g, 0.9 mmol), tri-tert-butylphosphine (0.27 g, 0.13 mmol) and toluene (100 mL) was heated to 120° C. and reacted overnight. After the end of the reaction, water (100 mL) was added, extraction was carried out with dichloromethane (100 ml*3), an organic phase was evaporated under a reduced pressure, residues were subjected to silica gel column chromatographic separation to obtain 5.5 g of light yellow solid with a yield of 87%. HRMS (ESI) (m/z): 733.3351 [M+H]⁺.

### Synthesis of Compound 1d

Under nitrogen protection, a mixture of compound 1c (3.2 g, 4.4 mmol), triethyl orthoformate (20 mL), ammonium hexafluorophosphate (4.3 g, 26.2 mmol) and acid salt (0.2 mL) was heated to 80° C., stirred and reacted for 24 h, and 3.5 g of product was obtained by filtration, with a yield of 77%. HRMS (ESI) (m/z): 377.1550 [M/2−PF6]⁺.

### Synthesis of Complex 1

Under nitrogen protection, the compound 1d (3.0 g, 2.9 mmol), Pt (COD) Cl2 (1.0 g, 3.5 mmol) and sodium acetate (0.7 g, 8.6 mmol) were added into the tetrahydrofuran solution (20 mL), and reacted at 120° C. for 24 h. After the end of the reaction, water (100 mL) was added, extraction was carried out with dichloromethane (100 ml*3), an organic phase was evaporated under a reduced pressure, and residues were subjected to silica gel column chromatographic separation obtain 0.42 g of light yellow solid with a yield of 15%. HRMS (ESI) (m/z): 946.2516 [M+H]⁺.

### Example 2: Preparation of Complex 5

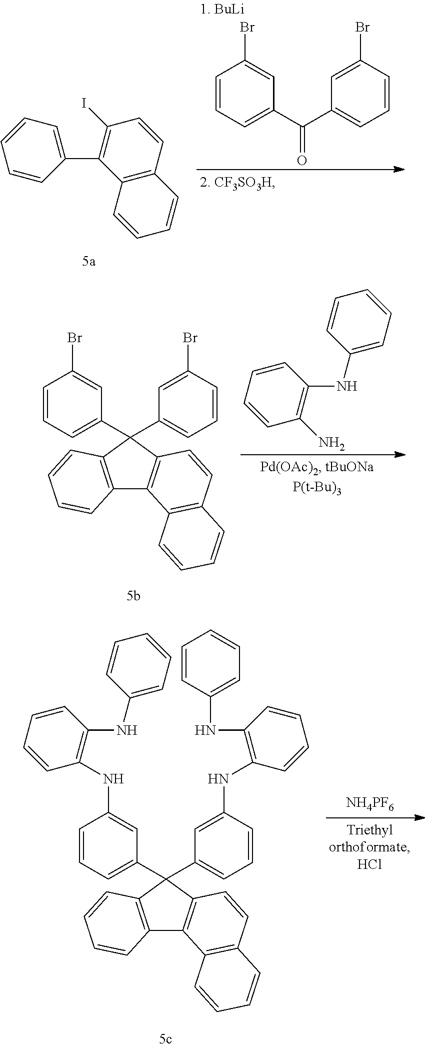

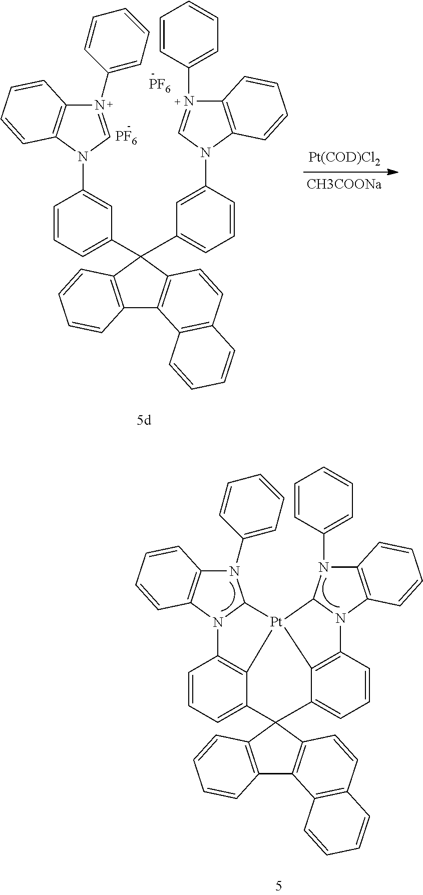

### Synthesis of Compound 5b

Compound 5b was prepared by referring to the method of compound 1b, and 6.6 g of product was obtained with a yield of 62%. HRMS (ESI) (m/z): 526.9822 [M+H]⁺.

### Synthesis of Compound 5c

Compound 5c was prepared by referring to the method of compound 1c, and 5.2 g of product was obtained with a yield of 79%. HRMS (ESI) (m/z): 733.3362 [M+H]⁺.

### Synthesis of Compound 5d

Compound 5d was prepared by referring to the method of compound 1d, and 3.5 g of product was obtained with a yield of 72%. HRMS (ESI) (m/z): 377.1550 [M/2−PF6]⁺.

### Synthesis of Complex 5

Complex 5 was prepared by referring to the method of compound 5d, and 0.32 g of product was obtained with a yield of 18%. HRMS (ESI) (m/z): 946.2526 [M+H]⁺.

### Example 3: Preparation of Complex 89

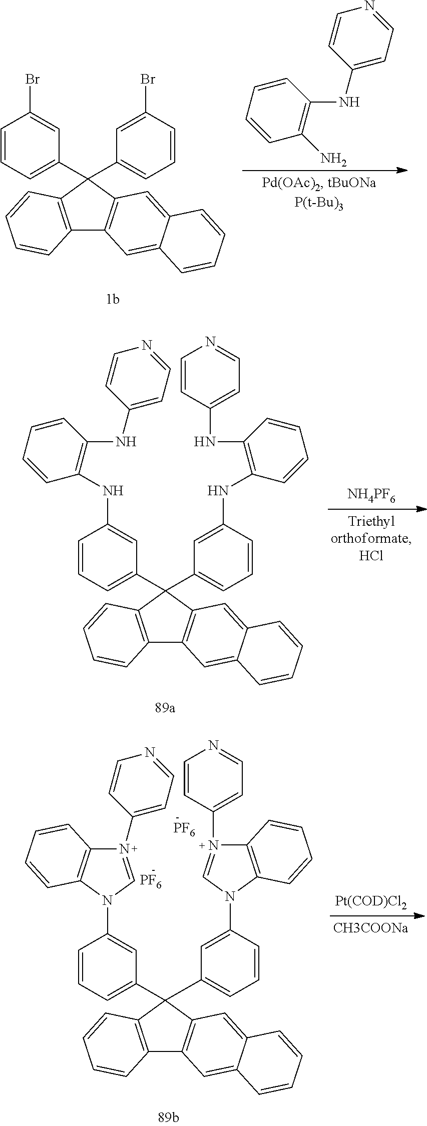

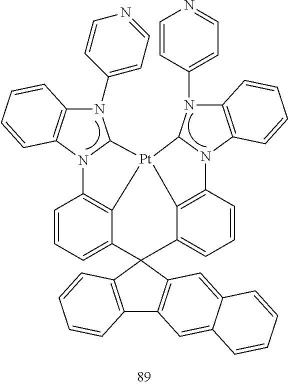

### Synthesis of Compound 89a

Compound 89a was prepared by referring to the method of compound 1c, and 6.2 g of product was obtained with a yield of 69%. HRMS (ESI) (m/z): 735.3251 [M+H]⁺.

### Synthesis of Compound 89b

Compound 89b was prepared by referring to the method of compound 1d, and 3.6 g of product was obtained with a yield of 73%. HRMS (ESI) (m/z): 378.1550 [M/2-PF6]⁺.

### Synthesis of Complex 89

Complex 89 was prepared by referring to the method of compound 5d, and 0.26 g of product was obtained with a yield of 13%. HRMS (ESI) (m/z): 948.2425 [M+H]⁺.

### Examples 4-6

An organic light-emitting diode was prepared using the complex light-emitting material of the present disclosure. The device structure is shown in FIGURE.

First, the transparent conductive ITO glass substrate 10 (with the anode 20 on it) was cleaned with a detergent solution and deionized water, ethanol, acetone, and deionized water in turn, and then treated with oxygen plasma for 30 s.

Then, HATCN was evaporated on the ITO to prepare a hole injection layer 30.

Then, HT was evaporated on the hole injection layer to form a hole transport layer 40 with a thickness of 40 nm.

Then, a light-emitting layer 50 was evaporated on a hole blocking layer, and the composition of the light-emitting layer was: platinum complex: BHI (base material)=6%: 100%, (the platinum complexes corresponding to Examples 4-6 were complexes 1, 5, 89, respectively).

Then, 40 nm thick ET was evaporated on the light-emitting layer as an electron transport layer 60.

Finally, 1 nm LiF was evaporated as an electron injection layer 70 and 100 nm Al was evaporated as a device cathode 80.

### Comparative Example 1

The device of Comparative example 1 was prepared using the same preparation method, using the compound Ref-Pt to replace the platinum complex in the above example.

The structural formulas of HATCN, HT, BH, ET and Ref-Pt in the device are as follows:

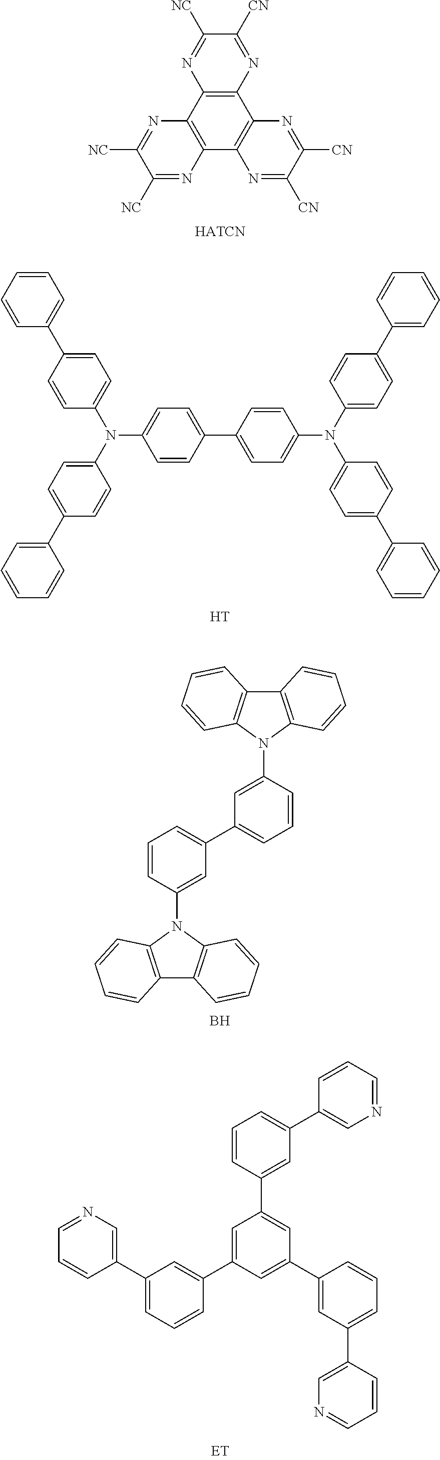

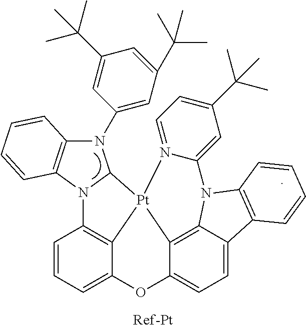

The device performance of the organic electroluminescent devices of Examples 4-6 and Comparative example 1 at a current density of 10 mA/cm² is listed in Table 1:

TABLE 1

| | | | | | Service |
|---|---|---|---|---|---|
| | | | | | life of |
| | | Driving | Luminous | Emitting | device |
| Device No. | Complex | voltage | efficiency | color | (LT70) |
| Comparative | Ref-Pt | 1 | 1 | blue | 1 |
| example 1 | | | | | |
| Example 4 | 1 | 0.96 | 1.12 | blue | 1.15 |
| Example 5 | 5 | 0.94 | 1.18 | blue | 1.23 |
| Example 6 | 89 | 0.98 | 1.21 | blue | 1.06 |

Remark: the device performance test uses Comparative example 1 as the benchmark, and all indicators are set to 1; LT70 represents the time corresponding to the device brightness decaying to 70% (initial brightness 1000 cd/m²).

It can be seen from the data in Table 1 that under the same conditions, the platinum complex material of the present disclosure is used in the organic light-emitting diode and emit deep blue light. Compared with the reference molecule Ref-Pt, the platinum complex material of the present disclosure has better luminous efficiency and service life of device, and has good industrialization potential.

The various embodiments described above are only examples and are not intended to limit the scope of the present disclosure. Various materials and structures in the present disclosure can be replaced by other materials and structures without departing from the spirit of the present disclosure. It should be understood that those skilled in the art can make many modifications and changes according to the ideas of the present disclosure without creative efforts. Therefore, any technical solution that a skilled person can obtain through analysis, reasoning or partial research based on the existing technology should be within the scope of protection limited by the claims.

1. A platinum complex, having a structure of formula (I): 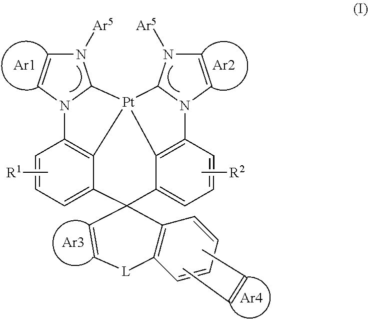
wherein:
L is selected from CR³R⁴, NR⁵, O, S or a single bond;
R¹ to R⁵ are independently selected from hydrogen, deuterium, halogen, amine, carbonyl group, carboxyl group, cyano group, phosphino, substituted or unsubstituted alkyl group with 1-20 carbon atoms, substituted or unsubstituted cycloalkyl group with 3-20 ring carbon atoms, substituted or unsubstituted alkenyl group with 2-20 carbon atoms, substituted or unsubstituted alkoxy group with 1-20 carbon atoms, substituted or unsubstituted aryl group with 6-30 carbon atoms, or substituted or unsubstituted heteroaryl group with 3-30 carbon atoms;
Ar¹ to Ar⁵ are independently selected from substituted or unsubstituted aromatic ring with 6-30 carbon atoms, or substituted or unsubstituted heteroaromatic ring with 3-30 carbon atoms;
a substitution is conducted by halogen, amine, cyano group or C1-C4 alkyl group; and
a heteroatom in the heteroaryl group or the heteroaromatic ring is at least one of N, S, and O.

2. The platinum complex according to claim 1, wherein R¹ to R⁵ are each independently selected from hydrogen, deuterium, halogen, amine, cyano group, substituted or unsubstituted alkyl group with 1-6 carbon atoms, substituted or unsubstituted cycloalkyl group with 3-6 ring carbon atoms, substituted or unsubstituted alkenyl group with 2-6 carbon atoms, substituted or unsubstituted alkoxy group with 1-6 carbon atoms, substituted or unsubstituted aryl group with 6-12 carbon atoms, or substituted or unsubstituted heteroaryl group with 3-6 carbon atoms; and
Ar¹ to Ar⁵ are independently selected from substituted or unsubstituted aromatic ring with 6-12 carbon atoms, or substituted or unsubstituted heteroaromatic ring with 3-12 carbon atoms.

3. The platinum complex according to claim 2, wherein R¹ to R⁵ are each independently selected from hydrogen, deuterium, halogen, cyano group, C1-C4 alkyl group, substituted or unsubstituted cycloalkyl group with 3-6 ring carbon atoms, substituted or unsubstituted aryl group with 6-12 carbon atoms, or substituted or unsubstituted heteroaryl group with 3-6 carbon atoms; and
Ar¹ to Ar⁵ are independently selected from substituted or unsubstituted aromatic ring with 6-12 carbon atoms, or substituted or unsubstituted heteroaromatic ring with 3-12 carbon atoms.

4. The platinum complex according to claim 3, wherein R¹ to R⁵ are each independently selected from hydrogen, deuterium, halogen, cyano group, methyl, isopropyl, isobutyl, tert-butyl, substituted or unsubstituted cyclopentyl, substituted or unsubstituted cyclohexyl, substituted or unsubstituted phenyl, substituted or unsubstituted pyridyl, substituted or unsubstituted pyrazinyl, or substituted or unsubstituted pyrimidinyl;
Ar¹ to Ar⁵ are independently selected from substituted or unsubstituted benzene ring, substituted or unsubstituted pyridine ring, substituted or unsubstituted pyrazine ring, substituted or unsubstituted pyrimidine ring, substituted or unsubstituted furan ring, substituted or unsubstituted thiophene ring, substituted or unsubstituted naphthalene ring, substituted or unsubstituted benzofuran ring, substituted or unsubstituted benzothiophene ring, substituted or unsubstituted thiazole ring, substituted or unsubstituted oxazole ring, substituted or unsubstituted pyrrole ring or substituted or unsubstituted imidazole ring; and
a substitution is conducted by cyano group or C1-C4 alkyl group.

5. The platinum complex according to claim 4, wherein in formula (I), R¹ to R⁵ are each independently selected from hydrogen, deuterium, fluorine, chlorine, methyl, tert-butyl, cyano group, substituted or unsubstituted cyclopentyl, substituted or unsubstituted cyclohexyl, or substituted or unsubstituted phenyl; and
Ar¹ to Ar⁵ are independently selected from substituted or unsubstituted benzene ring, substituted or unsubstituted pyridine ring, furan ring, thiophene ring, benzothiophene ring, benzofuran ring, or pyridine ring.

6. The platinum complex according to claim 1, wherein R¹ to R² are each independently selected from hydrogen, deuterium, chlorine, tert-butyl; and R³ to R⁵ are each independently selected from hydrogen, deuterium, tert-butyl, or phenyl; and
Ar¹, Ar², and Ar⁵ are independently selected from benzene ring, benzothiophene ring and benzofuran ring, and Ar³ and Ar⁴ are selected from benzene ring, pyridine ring, furan ring and thiophene ring.

7. The platinum complex according to claim 1, wherein R¹ to R² are each independently selected from hydrogen, deuterium, chlorine and tert-butyl, and L is a single bond; and
Ar¹ and Ar² are selected from benzene ring; Ar⁵ is selected from benzene ring, benzothiophene ring, benzofuran ring and pyridine ring; Ar³ is selected from benzene ring; and Ar⁴ is selected from the benzene ring, pyridine ring, furan ring and thiophene ring.

8. The platinum complex according to claim 1, selected from one of the following compounds:

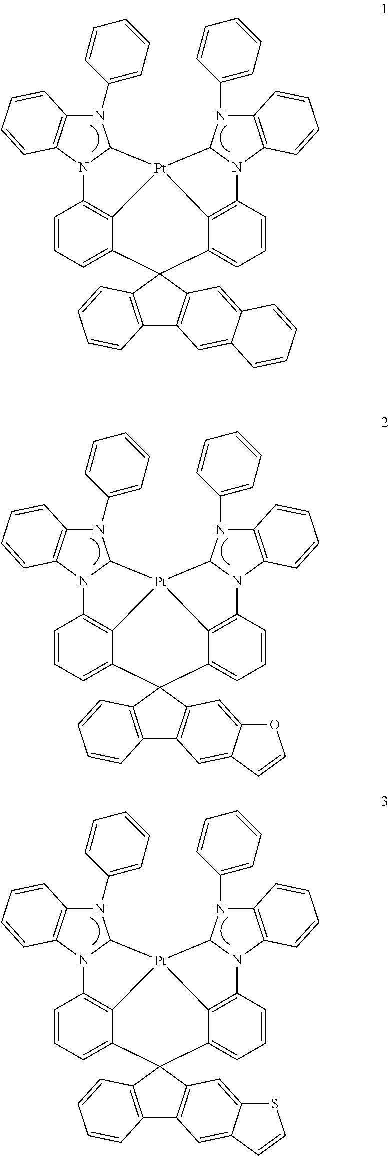

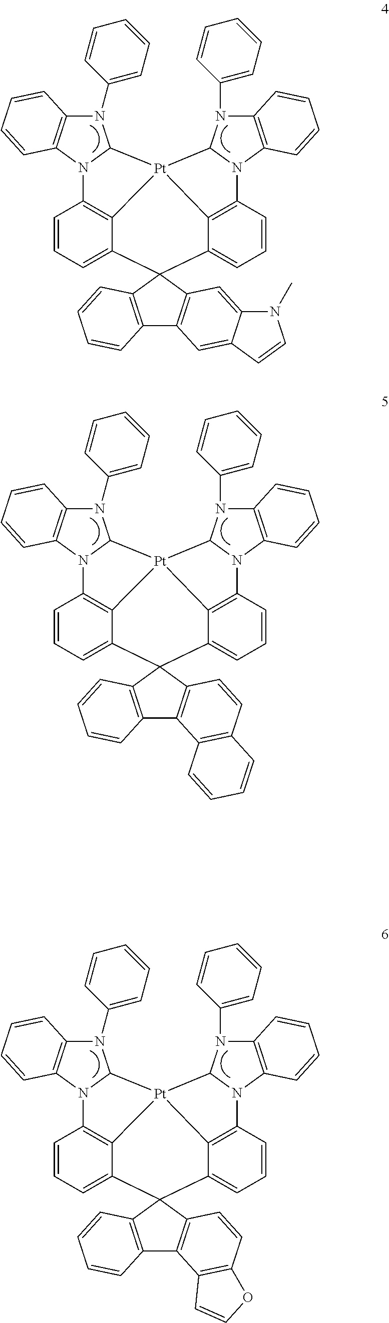

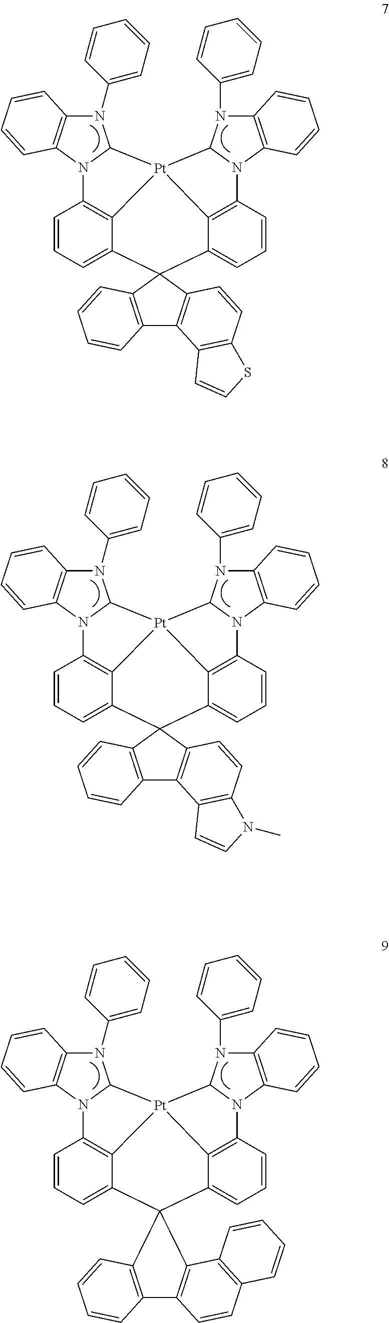

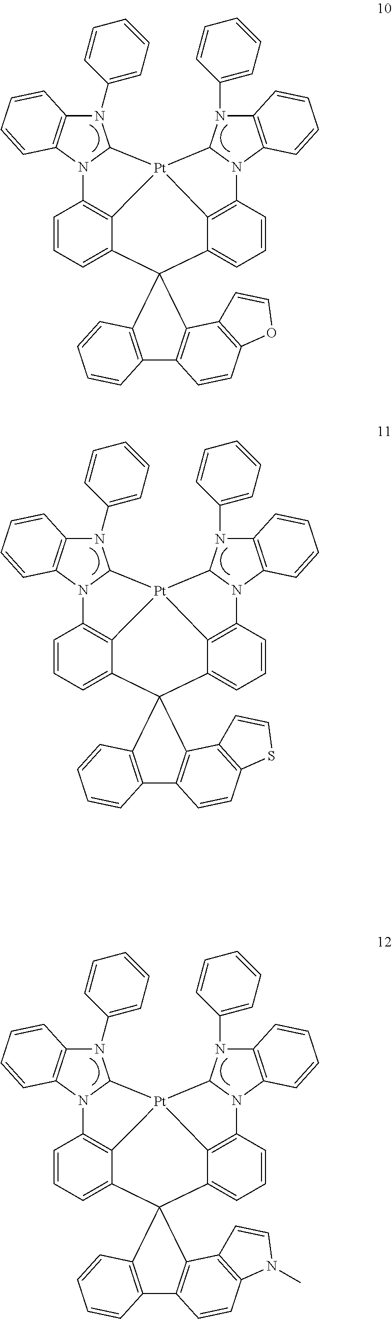

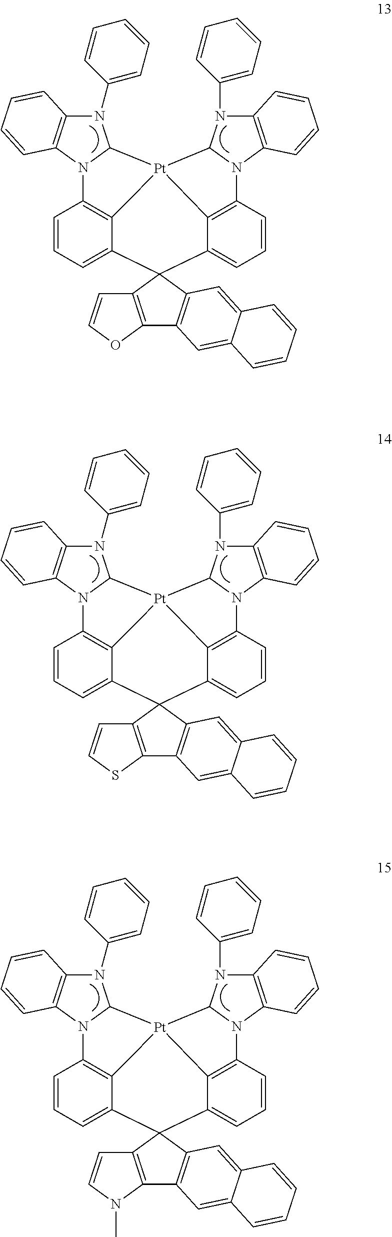

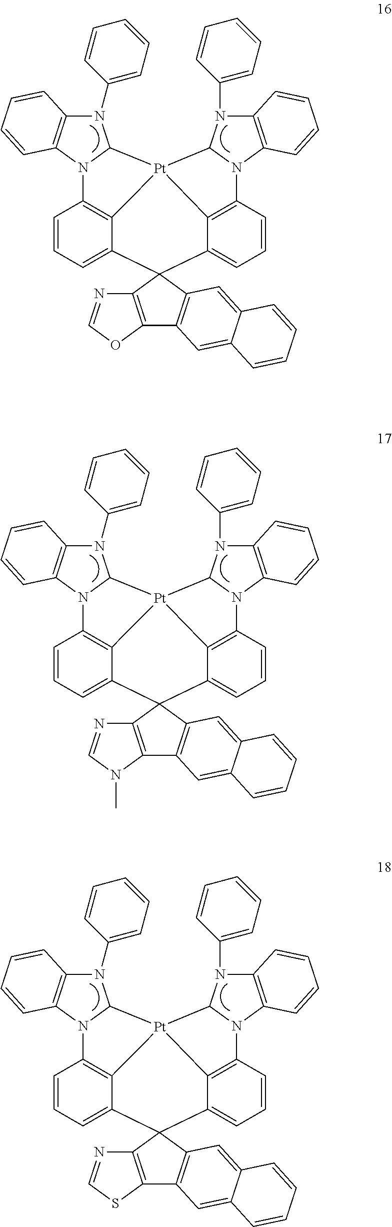

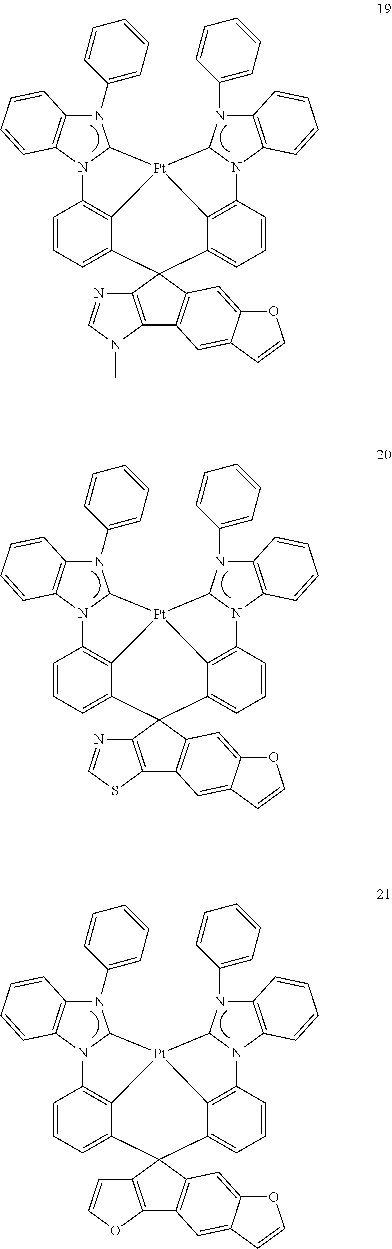

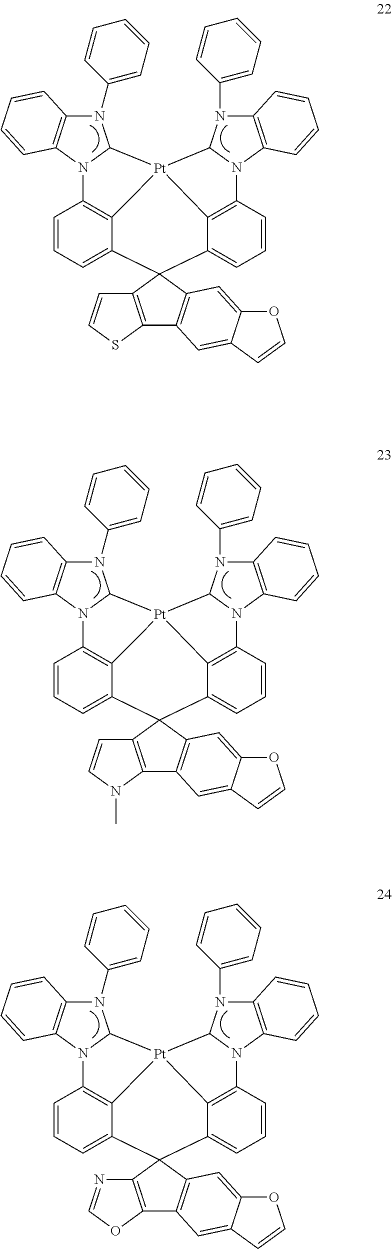

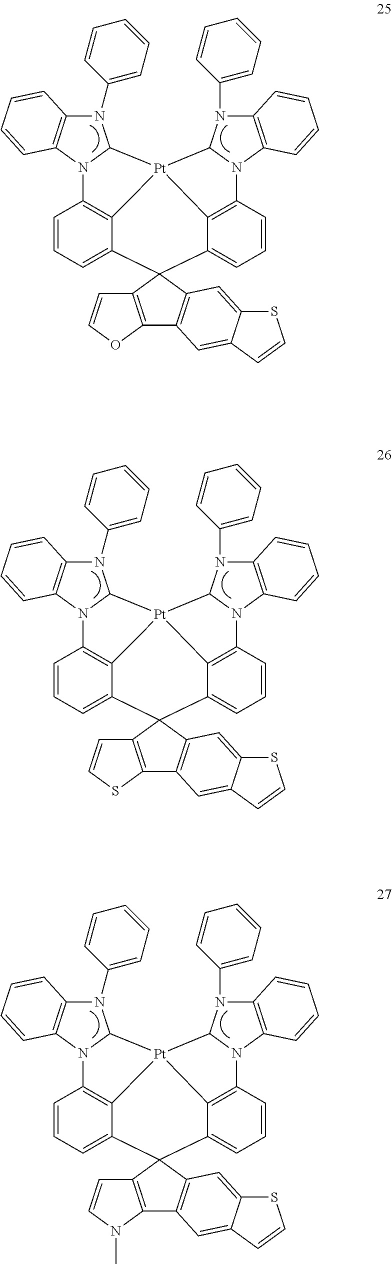

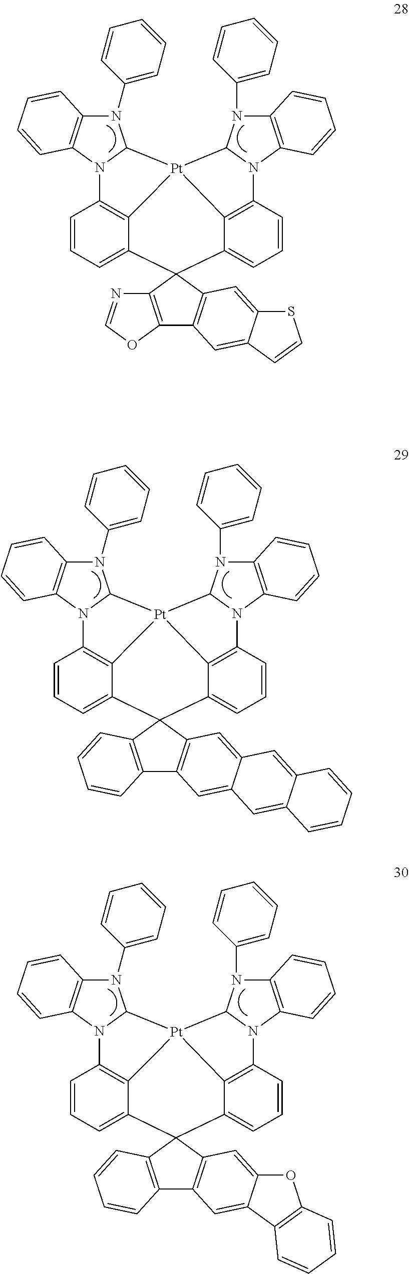

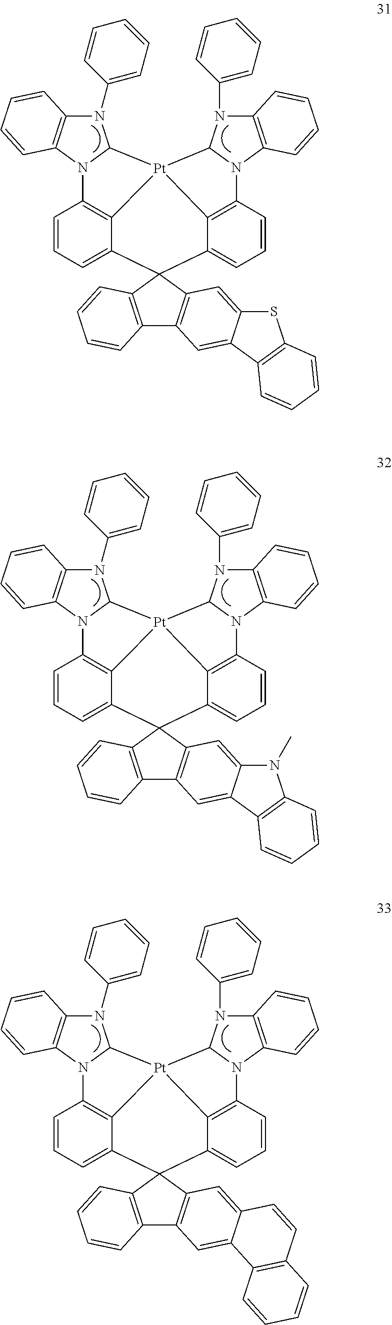

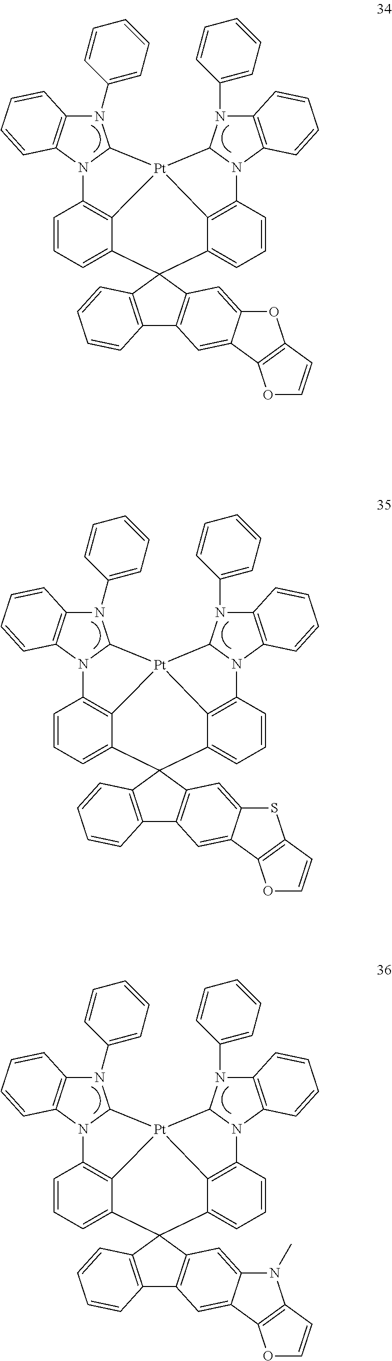

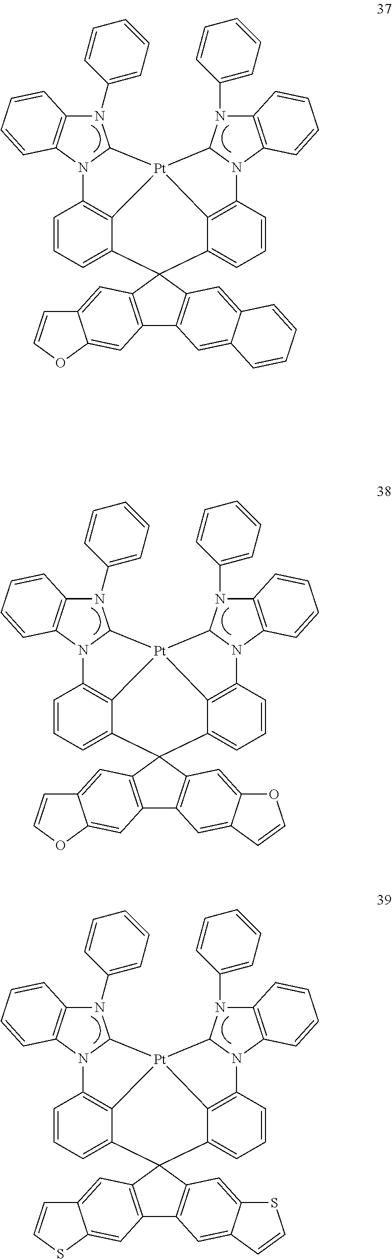

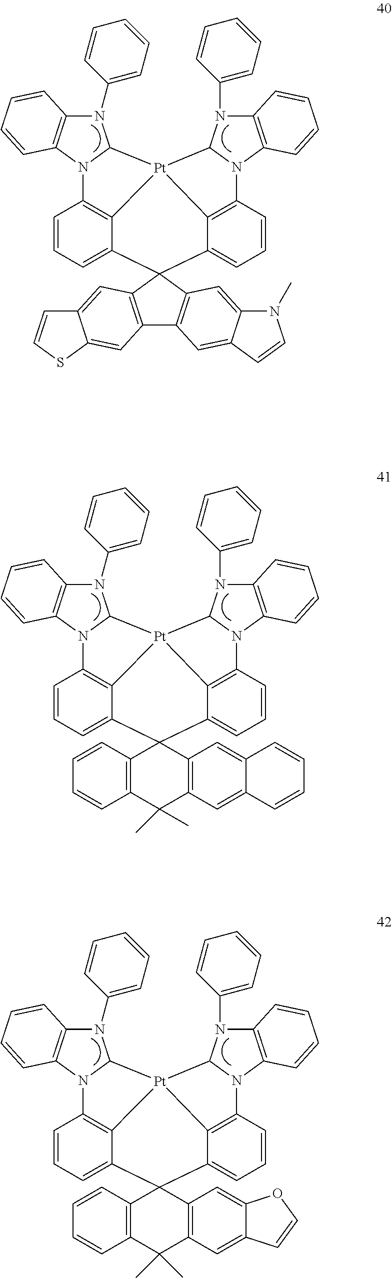

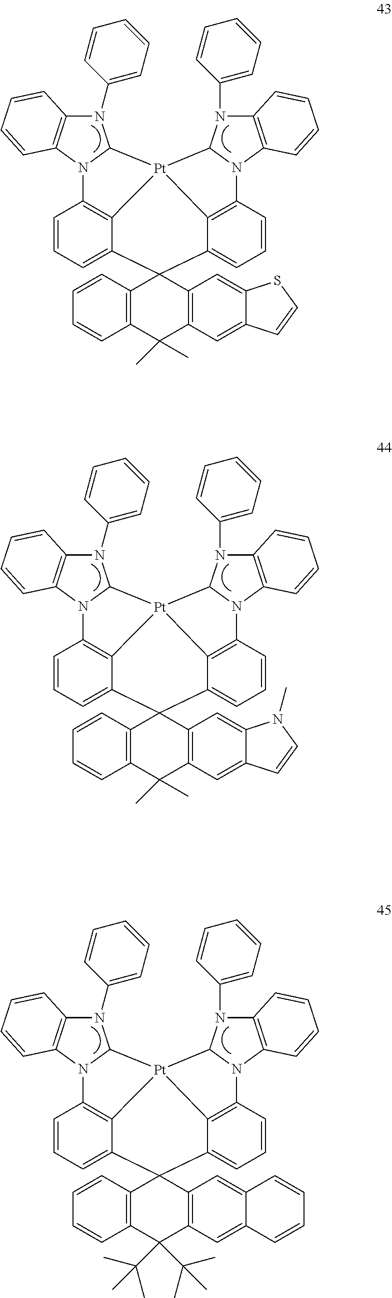

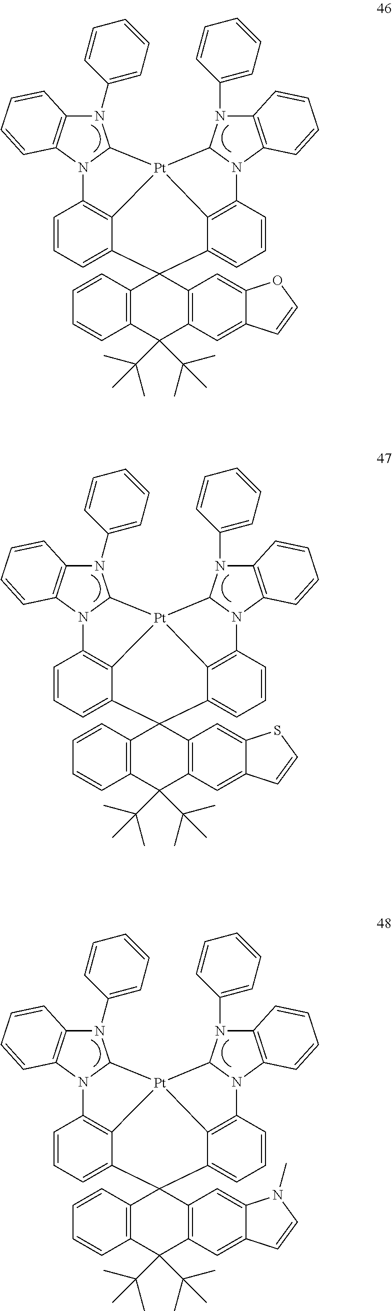

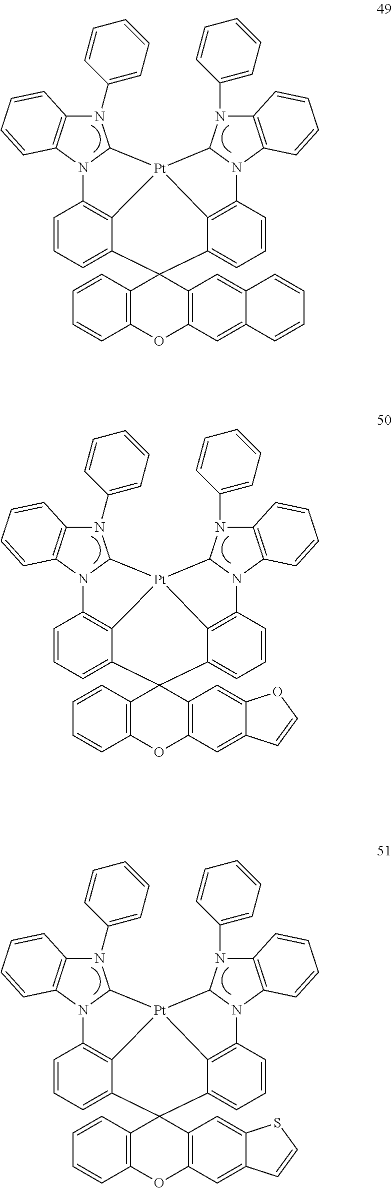

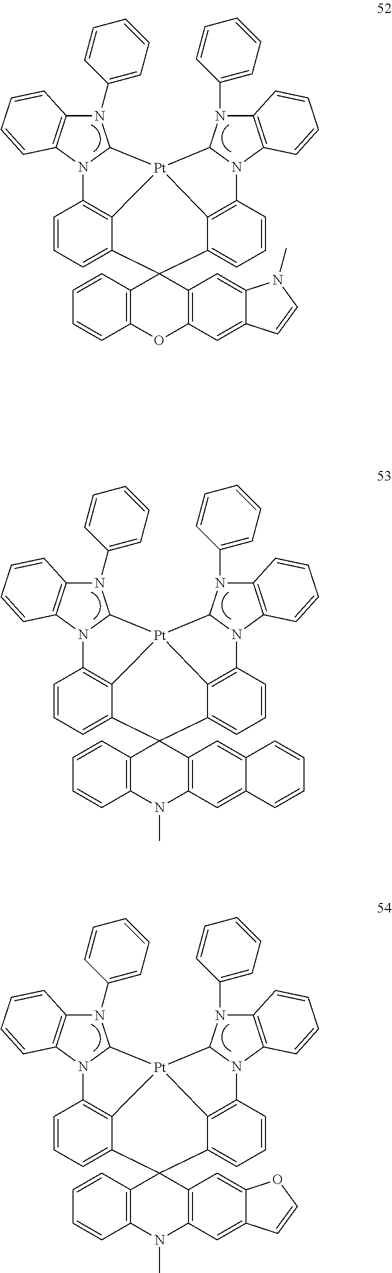

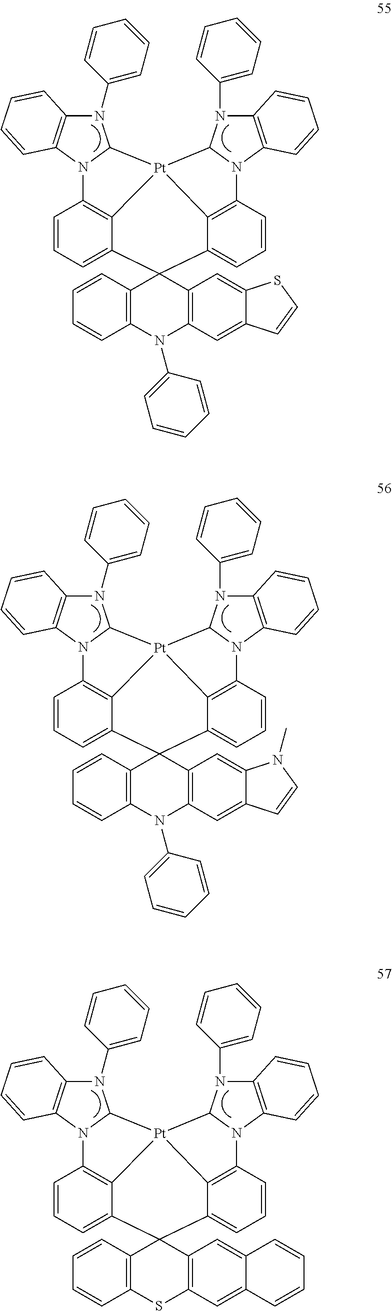

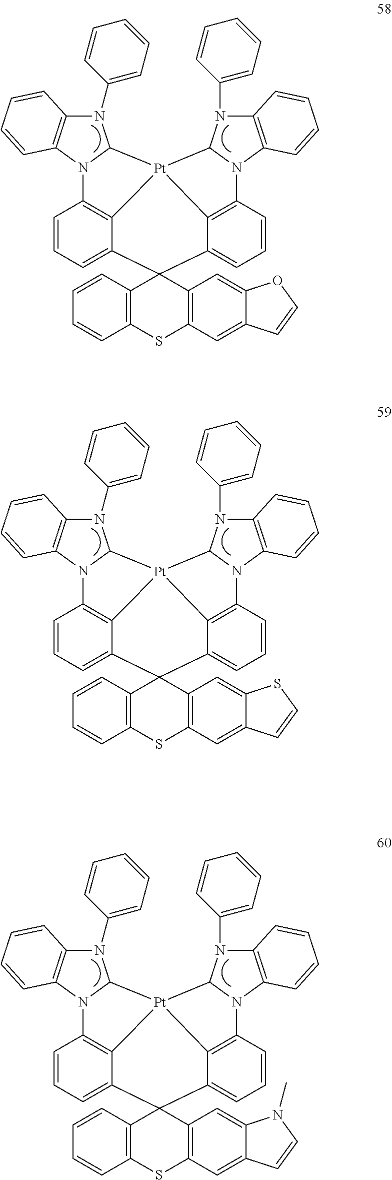

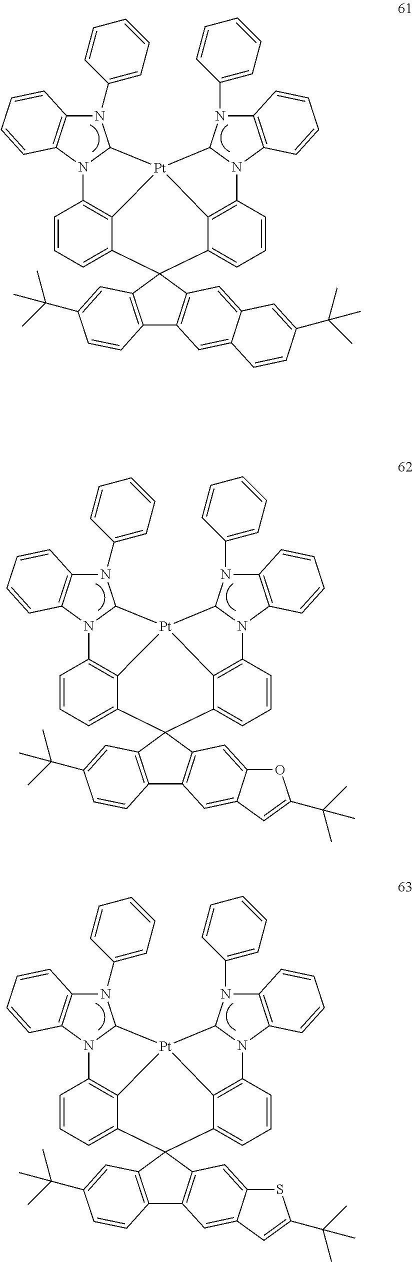

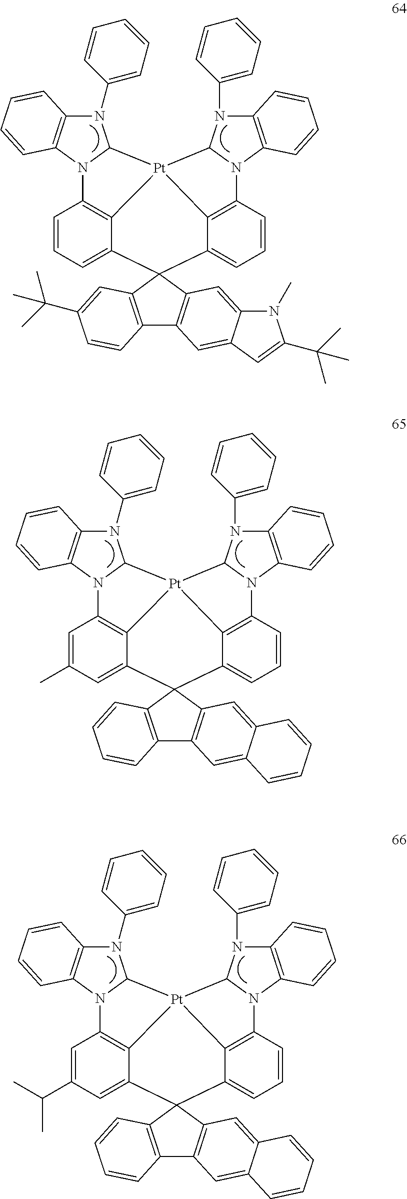

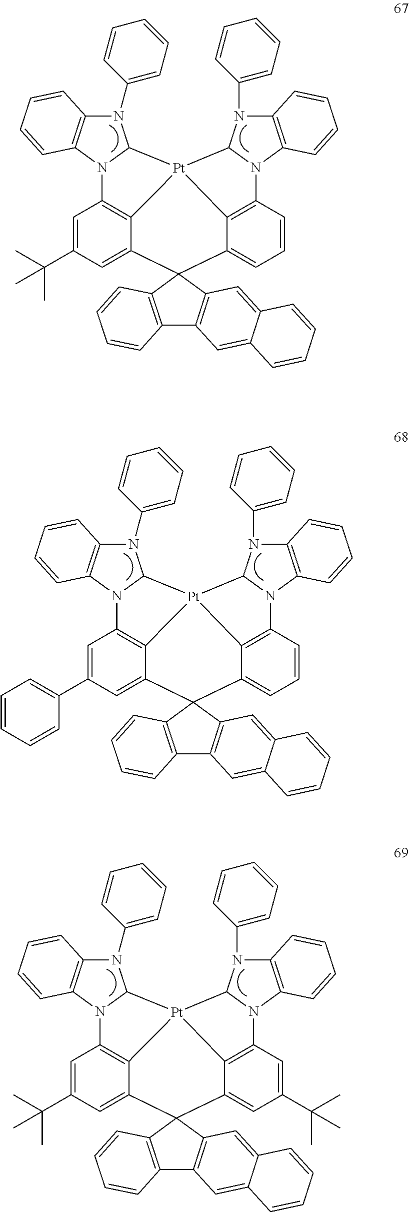

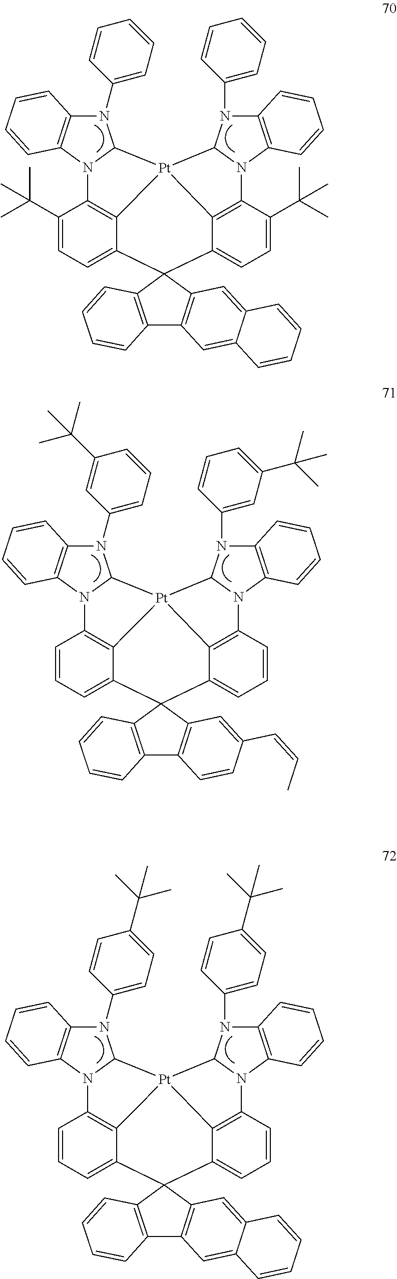

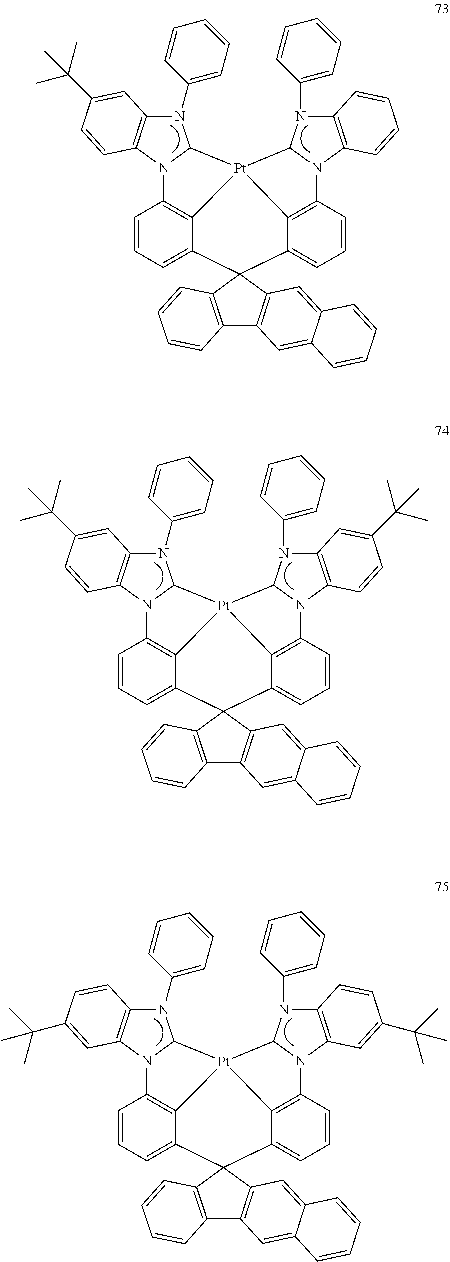

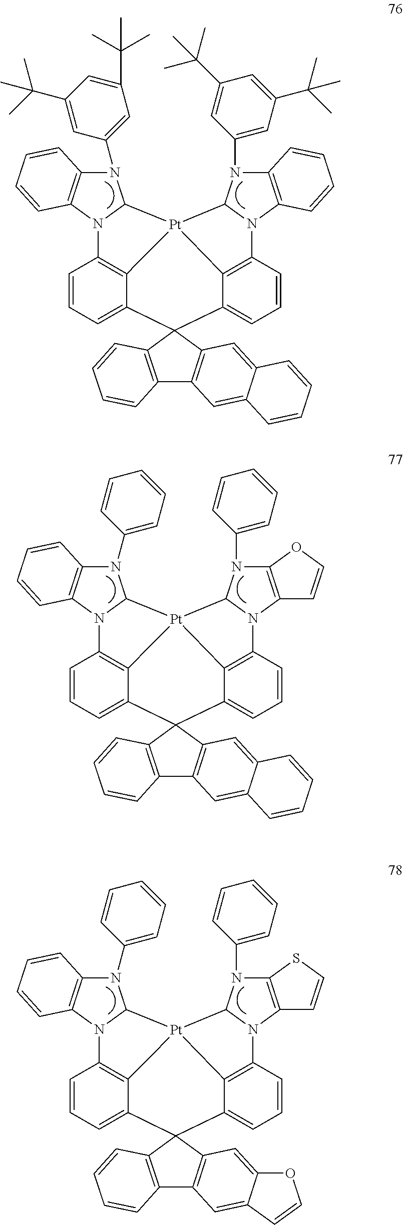

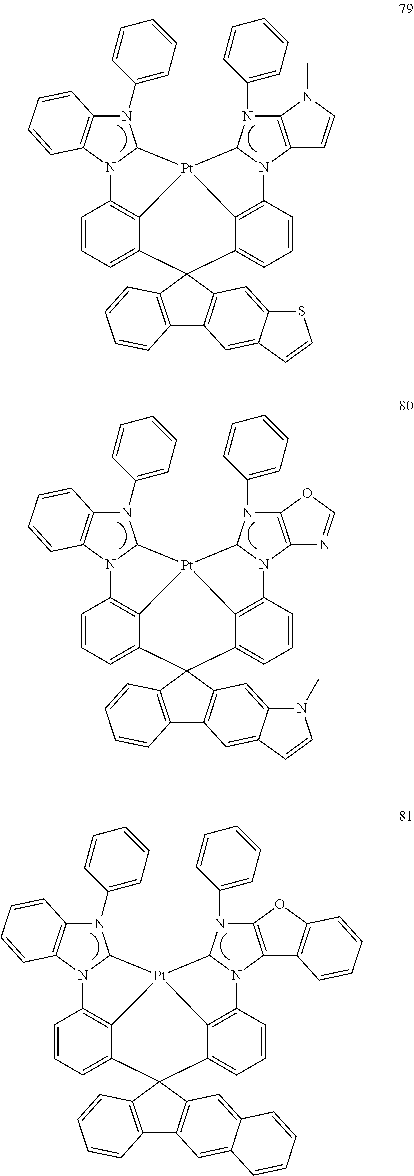

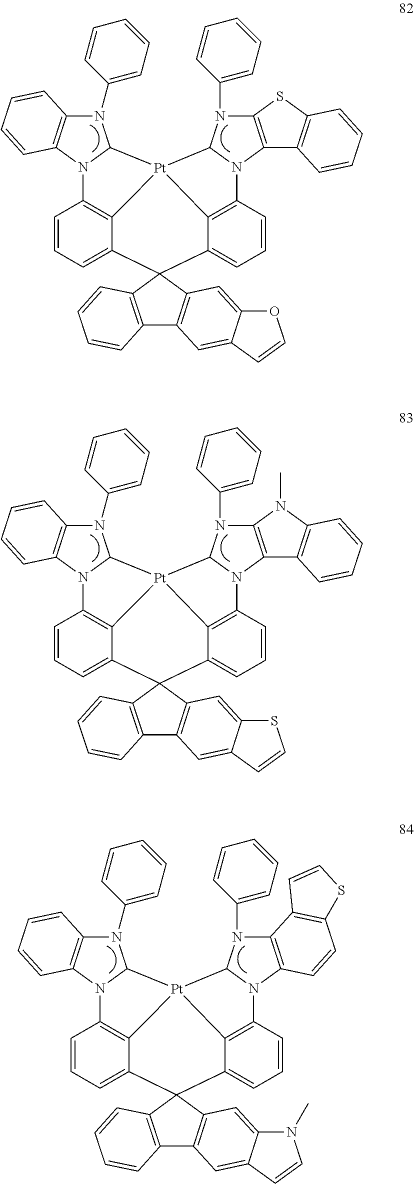

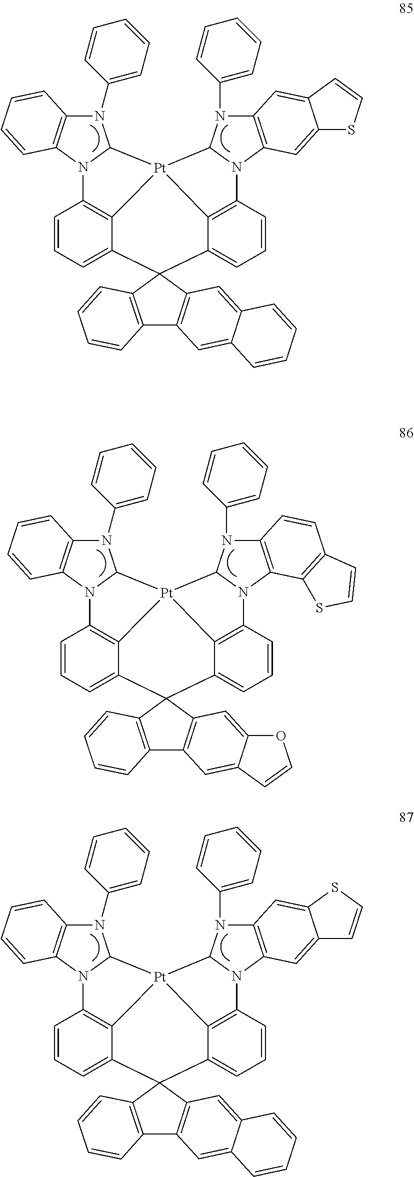

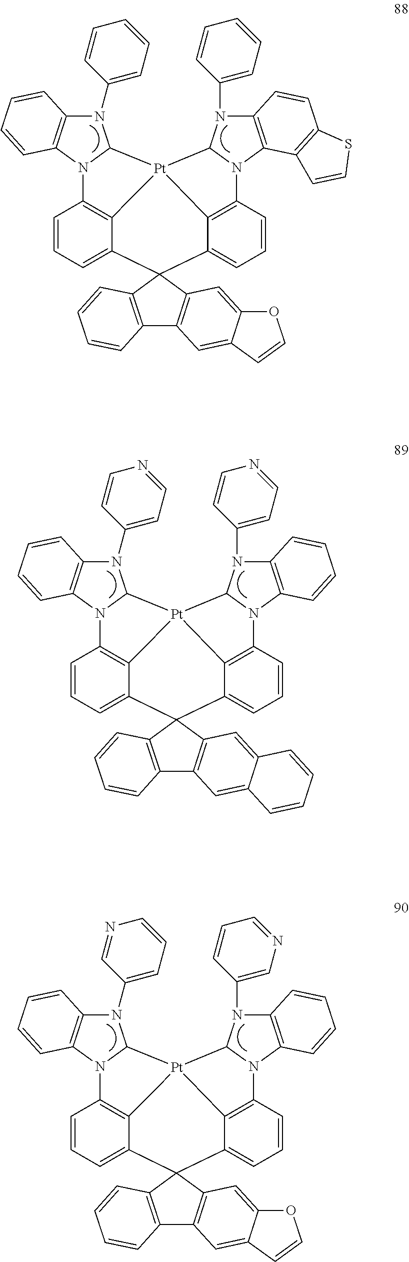

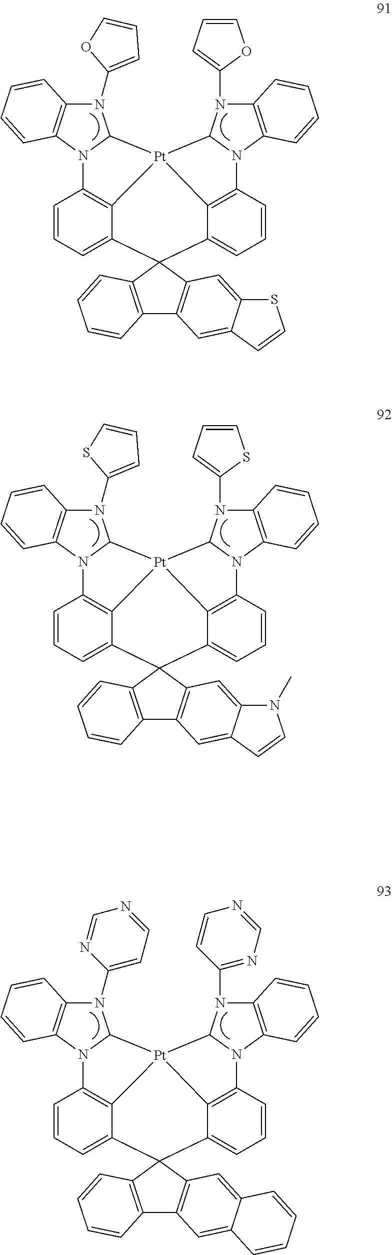

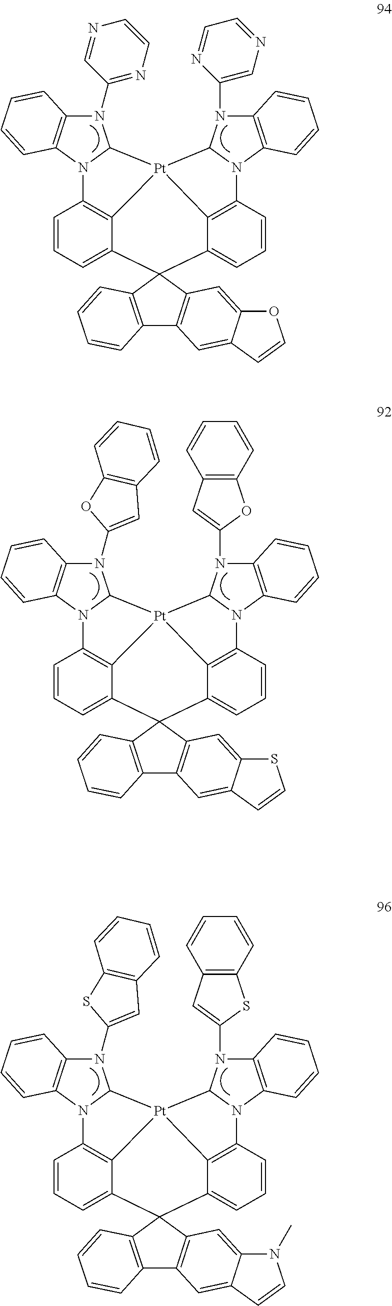

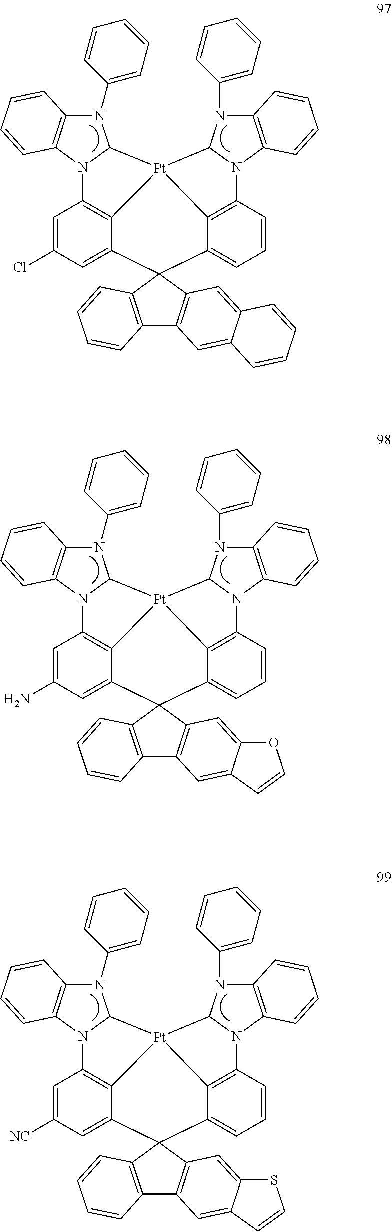

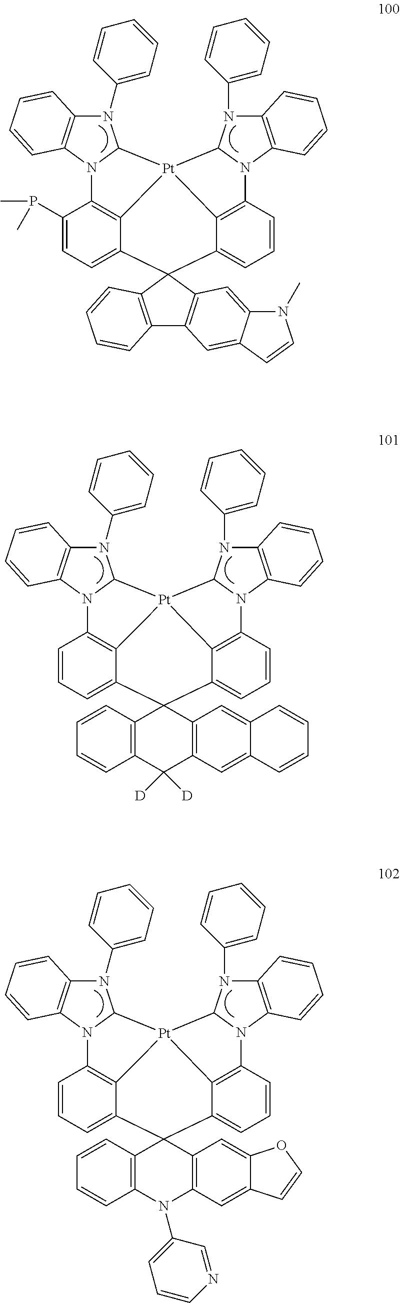

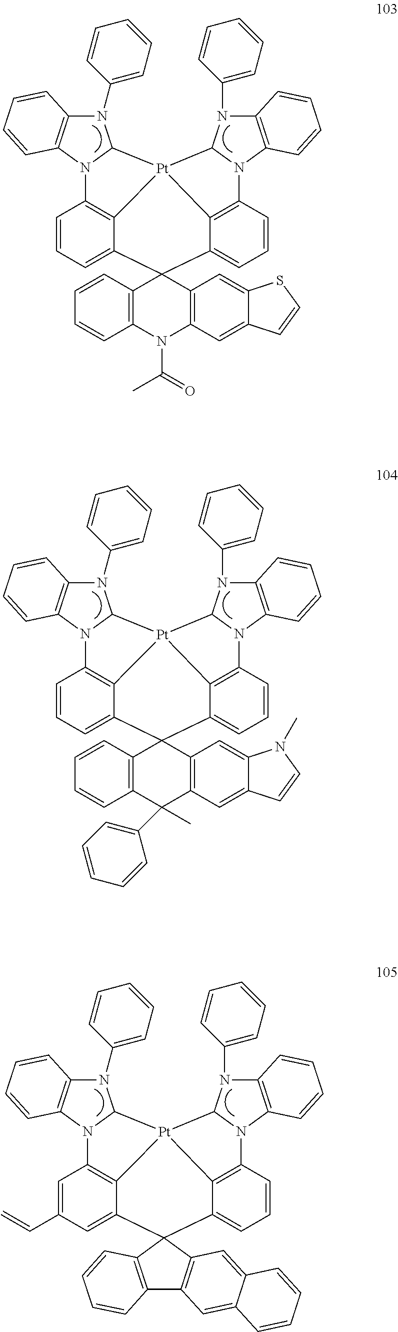

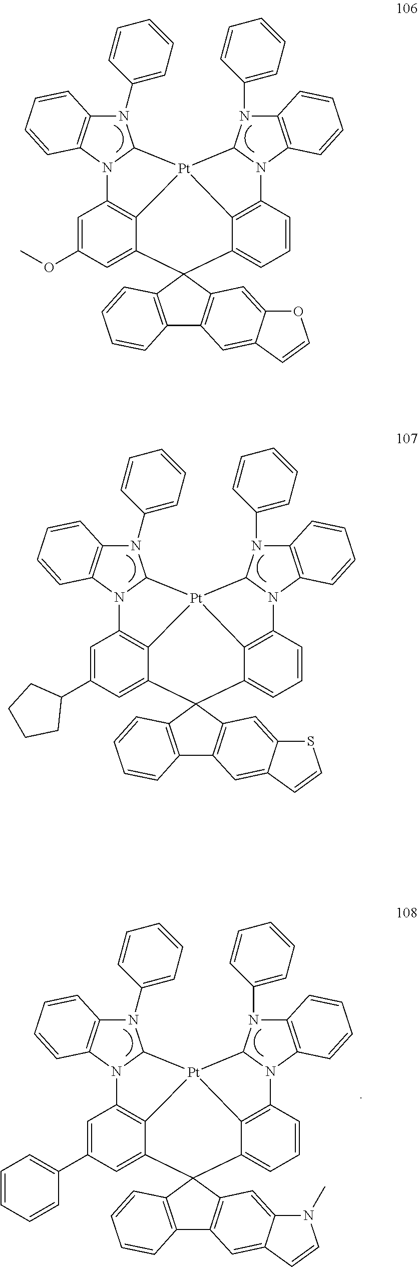

9. A precursor of the platinum complex according to claim 1, wherein the precursor has a structural formula as follows: 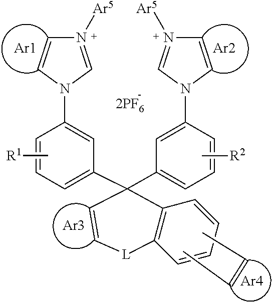
wherein L, R¹, R², Ar¹, Ar², Ar³, Ar⁴ and Ar⁵ are defined as formula (I).

10. An organic optoelectronic device comprising the platinum complex according to claim 1, wherein the organic optoelectronic device is an organic light-emitting diode, an organic thin film transistor, an organic photovoltaic device, a light-emitting electrochemical cell or a chemical sensor.

11. An organic light-emitting diode, comprising a cathode, an anode and an organic layer, wherein the organic layer is one or more of a hole injection layer, a hole transport layer, a light-emitting layer, a hole blocking layer, an electron injection layer and an electron transport layer, and wherein the organic layer contains the platinum complex according to claim 1.

12. The organic light-emitting diode according to claim 11, wherein the layer in which the platinum complex is located is the light-emitting layer.

13. An organic light-emitting diode, comprising a cathode, an anode and an organic layer, wherein the organic layer is one or more of a hole injection layer, a hole transport layer, a light-emitting layer, a hole blocking layer, an electron injection layer and an electron transport layer, and wherein the organic layer contains the platinum complex according to claim 2.

14. An organic light-emitting diode, comprising a cathode, an anode and an organic layer, wherein the organic layer is one or more of a hole injection layer, a hole transport layer, a light-emitting layer, a hole blocking layer, an electron injection layer and an electron transport layer, and wherein the organic layer contains the platinum complex according to claim 3.
